(19) United States
(12) Patent Application Publication
(10) Pub. No.: US 2010/0042438 A1
(43) Pub. Date: Feb. 18, 2010

(54) Methods and Systems for Personalized Action Plans

(75) Inventors: Stephen M. Moore, San Jose, CA (US); Michael A. Nierenberg, Palo Alto, CA (US); Sean E. George, Oakland, CA (US); Laurie A. Gomer, San Francisco, CA (US)

Correspondence Address: WILSON, SONSINI, GOODRICH & ROSATI, 650 PAGE MILL ROAD, PALO ALTO, CA, 94304-1050, US

(73) Assignee: Navigenics, Inc., Redwood Shores, CA (US)

(21) Appl. No.: 12/538,064

(22) Filed: Aug. 7, 2009

Related U.S. Application Data
(60) Provisional application No. 61/087,586, filed on Aug. 8, 2008.

(51) Int. Cl. G06Q 50/00; C12Q 1/68; C40B 20/00

(52) U.S. Cl. 705/3; 435/6; 506/2

(57) ABSTRACT

The present disclosure provides methods and systems for personal action plans based on an individual's genomic profile. Methods include assessing the association between an individual's genotype and at least one disease or condition and providing rating systems for an individual's action plan. Incentives to motivate and encourage people to improve their health and well-being are also disclosed herein.

### CROSS REFERENCE TO RELATED APPLICATION

This application claims priority to U.S. Provisional Application No. 61/087,586, filed Aug. 8, 2008, which application is incorporated herein by reference in its entirety.

### BACKGROUND

Genetic variations in the genome, such as single nucleotide polymorphisms (SNPs), mutations, deletions, insertions, repeats, microsatellites and others, are correlated to various phenotypes, such as a disease or condition. The genetic variations of an individual can be identified and correlated to determine the individual's predisposition to different phenotypes, creating a personalized phenotype profile.

An individuals' phenotype profile provides a personalized assessment of an individual's risk or likelihood of having a certain phenotype, and the individual may be interested in medical, as well as lifestyle options, for reducing or increasing a risk for a particular condition. An individual can benefit from a personalized action plan that integrates an individual's genomic profile, which can also further encompass non-genetic factors, such as past and current environmental and lifestyle factors.

Thus, a personalized action plan provides a customized approach for an individual, or their health care manager, to make informed and appropriate choices in promoting their health and well-being. Thus, there exists a need to provide individuals and their health care managers with a system that integrates their personal genomic profile to make appropriate medical and lifestyle choices into an action plan that is easy to follow, and can optionally, have incentives to motivate an individual to follow their personalized action plan. The embodiments disclosed herein satisfy these needs and provides related advantages as well.

### SUMMARY

The present disclosure provides methods and systems for generating personalized action plans based on an individual's genomic profile. Also provided are methods and systems for motivating individuals to lead healthier lifestyles, including methods that promote individuals in pursuing their action plans.

Described herein is a rating system for a variety of recommendations in a personalized action plan, wherein each of the recommendations is given a rating. The ratings can be generated or determined by a computer. Each rating corresponds to a rating given to an individual, wherein the rating given to an individual is based on a genomic profile of the individual. The rating given to the individual can be based on a Genetic Composite Index (GCI) or GCI Plus score of the individual. In some embodiments, the ratings are generated by a computer, based on a GCI or GCI Plus score that determined by the computer. The computer can then output the rating to the individual or a health care manager of the individual. The genomic profile can be obtained by amplifying a genetic sample from the individual, using a high density DNA microarray, PCR-based method, such as real-time PCR, or a combination thereof.

The rating can be a number, color, letter, or combination thereof, and the rating can be for a variety of recommendations, such as, but not limited to, one or more non-pharmaceutical recommendations. The non-pharmaceutical recommendation can be an exercise regimen, exercise activity, dietary plan, or combinations thereof. The non-pharmaceutical recommendation can also be nutrients, such as types of foods, vitamins, and the like. Furthermore, the rating can be part of rating system that is represented by a binary system, for example, a rating may be one of two designations.

Also disclosed herein is a method of providing a rating for recommendations in a personalized action plan to an individual comprising obtaining a genomic profile of the individual and determining at least one rating for the individual, wherein the rating is based on the genomic profile. In some embodiments, the method of providing a rating for recommendations in a personalized action plan to an individual comprises generating a GCI or GCI Plus score for the individual and determining at least one rating for the individual, wherein the rating is based on a GCI or GCI Plus score.

Also provided herein, is a method for motivating an individual to improve their health comprising obtaining a genomic profile for said individual, generating a personalized action plan for the individual, associating at least one incentive for the individual with an achievement of a recommendation on the personalized action plan, and granting to the individual the incentive when the achievement is accomplished. In some embodiments, the method of motivating an individual to improve their health comprises obtaining a genomic profile for said individual, generating at least one GCI or GCI Plus score for the individual, associating at least one incentive for the individual with an improvement of at least one GCI or GCI Plus score, and granting to the individual an incentive when the improvement is achieved. In some embodiments, the personalized action plan is generated or determined by a computer. For example, a computer can generate a GCI or GCI Plus score for an individual, and then use the GCI or GCI Plus score to generate a personalized action plan. The personalized action plan can then be outputted by the computer to the individual or a health care manager of the individual.

In some embodiments, the incentive is provided by an employer, friend, or family member. Thus, in some embodiments, the individual is an employee, and the incentive may be a contribution by an employer of said individual to a health savings account, extra vacation days, or increased employer subsidy for said individual's medical plan.

The incentive may also be cash, a pharmaceutical product, a health product, a health club membership, a medical follow-up, a medical device, an updated GCI or GCI Plus score, an updated personalized action plan, or membership to an on-line community. In some embodiments, the incentive is a discount, subsidy or reimbursement for a pharmaceutical product, a health product, a health club membership, a medical follow-up, a medical device, an updated GCI or GCI Plus score, an updated personalized action plan, or a membership to an on-line community. In yet other embodiments, the incentive is the support obtained through an on-line community.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the embodiments disclosed herein are set forth with particularity in the appended claims. A better understanding of the features and advantages of the disclosure will be obtained by reference to the following detailed description that sets forth illustrative embodiments, in which the principles of the disclosure herein are utilized, and the accompanying drawings of which:

FIG. 1 is a graph of the Receiver Operating Characteristic (ROC) curves for Crohn's Disease. The bottom line corresponds to random expectation and the top line corresponds to theoretical expectations when the genetic variable is known. The first middle line corresponds to GCI while the second middle line corresponds to the curve obtained by logistic regression.

FIG. 2 is a graph of the ROC curves for Type 2 Diabetes. The bottom line corresponds to random expectation and the top line corresponds to theoretical expectations when the genetic variable is known. The first middle line corresponds to GCI while the second middle line corresponds to the curve obtained by logistic regression.

FIG. 3 is a graph of the ROC curves for Rheumatoid Arthritis. The bottom line corresponds to random expectation and the top line corresponds to theoretical expectations when the genetic variable is known. The first middle line corresponds to GCI while the second middle line corresponds to the curve obtained by logistic regression.

FIG. 4 represents a rating system for an individual based on their genomic profile. A) represents a food rating in a personalized action plan for an individual predisposed to colon cancer and diabetes; B) represents a plain burger with no bun using this rating system; C) represents broccoli using this rating system; and D) represents an apple using this rating system.

FIG. 5 represents a schematic of a system for the analysis and transmission of genomic and phenotype profiles, and personalized action plans, over a network

### DETAILED DESCRIPTION

Disclosed herein are methods and systems for generating personalized action plans based on an individual's genomic profile. Also provided are methods and systems for motivating individuals to lead healthier lifestyles, including methods that promote individuals in pursuing their action plans.

### Genomic Profile

An individual's genomic profile contains information about an individual's genes based on genetic variations or markers. Genetic variations can form genotypes, which make up genomic profiles. Such genetic variations or markers include, but are not limited to, single nucleotide polymorphisms (SNPs), single and/or multiple nucleotide repeats, single and/or multiple nucleotide deletions, microsatellite repeats (small numbers of nucleotide repeats with a typical 5-1,000 repeat units), di-nucleotide repeats, tri-nucleotide repeats, sequence rearrangements (including translocation and duplication), copy number variations (both loss and gains at specific loci), and the like. Other genetic variations include chromosomal duplications and translocations, as well as centromeric and telomeric repeats.

Genotypes may also include haplotypes and diplotypes. In some embodiments, genomic profiles may have at least 100,000, 300,000, 500,000, or 1,000,000 genotypes. In some embodiments, the genomic profile may be substantially the complete genomic sequence of an individual. In other embodiments, the genomic profile is at least 60%, 80%, or 95% of the complete genomic sequence of an individual. The genomic profile may be approximately 100% of the complete genomic sequence of an individual. Genetic samples that contain the targets include, but are not limited to, unamplified genomic DNA or RNA samples or amplified DNA (or cDNA). The targets may be particular regions of genomic DNA that contain genetic markers of particular interest.

To obtain a genomic profile, a genetic sample of an individual can be isolated from a biological sample of an individual. The biological sample includes samples from which genetic material, such as RNA and/or DNA, may be isolated. Such biological samples can include, but not be limited to, blood, hair, skin, saliva, semen, urine, fecal material, sweat, buccal, and various bodily tissues. Tissues samples may be directly collected by the individual, for example, a buccal sample can be obtained by the individual taking a swab against the inside of their cheek. Other samples such as saliva, semen, urine, fecal material, or sweat, may also be supplied by the individual themselves. Other biological samples may be taken by a health care specialist, such as a phlebotomist, nurse or physician. For example, blood samples may be withdrawn from an individual by a nurse. Tissue biopsies may be performed by a health care specialist, and commercial kits are also readily available to health care specialists to efficiently obtain samples. A small cylinder of skin may be removed or a needle may be used to remove a small sample of tissue or fluids.

Sample collection kits can also be provided to individuals. The kits can contain sample collection containers for the individual's biological sample. The kit may also provide instructions for an individual to directly collect their own sample, such as how much hair, urine, sweat, or saliva to provide. The kit may also contain instructions for an individual to request tissue samples to be taken by a health care specialist. The kit may include locations where samples may be taken by a third party, for example, kits may be provided to health care facilities who in turn collect samples from individuals. The kit may also provide return packaging for the sample to be sent to a sample processing facility, where genetic material is isolated from the biological sample.

A genetic sample of DNA or RNA can be isolated from a biological sample according to any of several well-known biochemical and molecular biological methods, see, e.g., Sambrook, et al., Molecular Cloning: A Laboratory Manual (Cold Spring Harbor Laboratory, New York) (1989). There are also several commercially available kits and reagents for isolating DNA or RNA from biological samples, such as, but not limited to, those available from DNA Genotek, Gentra Systems, Qiagen, Ambion, and other suppliers. Buccal sample kits are readily available commercially, such as the MasterAmp™ Buccal Swab DNA extraction kit from Epicentre Biotechnologies, as are kits for DNA extraction from blood samples such as Extract-N-Amp™ from Sigma Aldrich. DNA from other tissues may be obtained by digesting the tissue with proteases and heat, centrifuging the sample, and using phenol-chloroform to extract the unwanted materials, leaving the DNA in the aqueous phase. The DNA can then be further isolated by ethanol precipitation.

For example, genomic DNA can be isolated from saliva, using a DNA self collection kit from DNA Genotek. An individual can collect a specimen of saliva for clinical processing using the kit and the sample can conveniently be stored and shipped at room temperature. After delivery of the sample to an appropriate laboratory for processing, DNA is isolated by heat denaturing and protease digesting the sample, typically using reagents supplied by the collection kit supplier at 50° C. for at least one hour. The sample is next centrifuged, and the supernatant is ethanol precipitated. The DNA pellet is suspended in a buffer appropriate for subsequent analysis.

RNA may be used as the genetic sample, for example, genetic variations that are expressed can be identified from mRNA. mRNA includes, but is not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a DNA amplified from the cDNA, an RNA transcribed from the amplified DNA, etc., are all derived from the mRNA transcript. RNA can be isolated from any of several bodily tissues using methods known in the art, such as isolation of RNA from unfractionated whole blood using the PAXgene™ Blood RNA System available from PreAnalytiX. Typically, mRNA is used to reverse transcribe cDNA, which is then used or amplified for gene variation analysis.

Prior to genomic profile analysis, a genetic sample may be amplified, either from DNA or cDNA reverse transcribed from RNA. DNA can be amplified by a number of methods, many of which employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86:1173-1177 (1989) and WO88/10315), self-sustained sequence replication (Guatelli et al., Proc. Nat. Acad. Sci. USA, 87:1874-1878 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) nucleic acid sequence based amplification (NASBA), rolling circle amplification (RCA), multiple displacement amplification (MDA) (U.S. Pat. Nos. 6,124,120 and 6,323,009) and circle-to-circle amplification (C2CA) (Dahl et al. Proc. Natl. Acad. Sci 101:4548-4553 (2004)). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 5,409,818, 4,988,617, 6,063,603 and 5,554,517 and in U.S. Ser. No. 09/854,317, each of which is incorporated herein by reference.

Generation of a genomic profile can be performed using any of several methods. Several methods are known in the art to identify genetic variations, and include, but are not limited to, DNA sequencing by any of several methodologies, PCR based methods, fragment length polymorphism assays (restriction fragment length polymorphism (RFLP), cleavage fragment length polymorphism (CFLP)) hybridization methods using an allele-specific oligonucleotide as a template (e.g., TaqMan assays and microarrays, further described herein), methods using a primer extension reaction, mass spectrometry (such as, MALDI-TOF/MS method), and the like, such as described in Kwok, Pharmocogenomics 1:95-100 (2000). Other methods include invader methods, such as monoplex and biplex invader assays (e.g. available from Third Wave Technologies, Madison, Wisc. and described in Olivier et al., Nucl. Acids Res. 30:e53 (2002)).

For example, a high density DNA array can be used to generate a genomic profile. Such arrays are commercially available from Affymetrix and Illumina (see Affymetrix GeneChip® 500K Assay Manual, Affymetrix, Santa Clara, Calif. (incorporated by reference); Sentrix® humanHap650Y genotyping beadchip, Illumina, San Diego, Calif.). A high density array can be used to generate a genomic profile that comprises genetic variations that are SNPs. For example, a SNP profile can be generated by genotyping more than 900,000 SNPs using the Affymetrix Genome Wide Human SNP Array 6.0. Alternatively, more than 500,000 SNPs through whole-genome sampling analysis may be determined by using the Affymetrix GeneChip Human Mapping 500K Array Set. In these assays, a subset of the human genome is amplified through a single primer amplification reaction using restriction enzyme digested, adaptor-ligated human genomic DNA. Typically, the amplified DNA is then fragmented and the quality of the sample determined prior denaturing and labeling the sample for hybridization to a microarray with DNA probes at specific locations on a coated quartz surface. The amount of label that hybridizes to each probe as a function of the amplified DNA sequence is monitored, thereby yielding sequence information and resultant SNP genotyping.

Use of high density arrays is well known in the arts, and if obtained commercially, is carried out according to the manufacturer's directions. For example, use of Affymetrix GeneChip can involve digesting isolated genomic DNA with either a NspI or StyI restriction endonuclease. The digested DNA is then ligated with a NspI or StyI adaptor oligonucleotide that respectively anneals to either the NspI or StyI restricted DNA. The adaptor-containing DNA following ligation is then amplified by PCR to yield amplified DNA fragments between about 200 and 1100 base pairs, as confirmed by gel electrophoresis. PCR products that meet the amplification standard are purified and quantified for fragmentation. The PCR products are fragmented with DNase I for optimal DNA chip hybridization. Following fragmentation, DNA fragments should be less than 250 base pairs, and on average, about 180 base pairs, as confirmed by gel electrophoresis. Samples that meet the fragmentation standard are then labeled with a biotin compound using terminal deoxynucleotidyl transferase. The labeled fragments are next denatured and then hybridized into a GeneChip 250K array. Following hybridization, the array is stained prior to scanning in a three step process consisting of a streptavidin phycoerythin (SAPE) stain, followed by an antibody amplification step with a biotinylated, anti-streptavidin antibody (goat), and final stain with streptavidin phycoerythin (SAPE). After labeling, the array is covered with an array holding buffer and then scanned, for example with a scanner such as the Affymetrix GeneChip Scanner 3000.

Analysis of data following scanning high density array can be performed according to the manufacturer's guidelines. For example, with the Affymetrix GeneChip, acquisition of raw data can be by use of the GeneChip Operating Software (GCOS) or by using Affymetrix GeneChip Command Console™. The aquisition of raw data is then followed by analysis with GeneChip Genotyping Analysis Software (GTYPE). Samples with a GTYPE call rate of less than a certain percentage may be excluded. For example, a call rate of less than approximately 70, 75, 80, 85, 90, or 95% may be excluded. Samples are then examined with BRLMM and/or SNiPer algorithm analyses. Samples with a BRLMM call rate of less than 95% or a SNiPer call rate of less than 98% are excluded. Finally, an association analysis is performed, and samples with a SNiPer quality index of less than 0.45 and/or a Hardy-Weinberg p-value of less than 0.00001 are excluded.

As an alternative to or in addition to DNA microarray analysis, genetic variations such as SNPs and mutations can be detected by other hybridization based methods, such as the use of TaqMan methods and variations thereof. TaqMan PCR, iterative TaqMan, and other variations of real time PCR (RT-PCR), such as those described in Livak et al, Nature Genet., 9, 341-32 (1995) and Ranade et al. Genome Res., 11, 1262-1268 (2001) can be used in the methods disclosed herein. In some embodiments, probes for specific genetic variations, such as SNPs, are labeled to form TaqMan probes. The probes are typically approximately at least 12, 15, 18 or 20 base pairs in length. They may be between approximately 10 and 70, 15 and 60, 20 and 60, or 18 and 22 base pairs in length. The probe is labeled to form reporter label, such as a fluorophore, at the 5′ end and a quencher of the label at the 3′ end. The reporter label may be any fluorescent molecule that has its fluorescence inhibited or quenched when in close proximity, such as the length of the probe, to the quencher. For example, the reporter label can be a fluorophore such as 6-carboxyfluorescein (FAM), tetracholorfluorescin (TET), or derivatives thereof, and the quencher tetramethylrhodamine (TAMRA), dihydrocyclopyrroloindole tripeptide (MGB), or derivatives thereof.

As the reporter fluorophore and quencher are in close proximity, separated by the length of the probe, the fluorescence is quenched. When the probe anneals to a target sequence, such as a sequence comprising a SNP in a sample, DNA polymerase with 5′ to 3′ exonuclease activity, such as Taq polymerase, can extend the primer and the exonuclease activity cleaves the probe, separating the reporter from the quencher, and thus the reporter can fluoresce. The process can be repeated, such as in RT-PCR. The TaqMan probe is typically complementary to a target sequence that is located between two primers that are designed to amplify a sequence. Thus, the accumulation of PCR product can be correlated to the accumulation of released fluorophore, as each probe can hybridize to newly generated PCR product. The released fluorophore can be measured and the amount of target sequence present can be determined. RT-PCR methods for high througput genotyping, such as in

Genetic variations can also be identified by DNA sequencing. DNA sequencing may be used to sequence a substantial portion, or the entire, genomic sequence of an individual. Traditionally, common DNA sequencing has been based on polyacrylamide gel fractionation to resolve a population of chain-terminated fragments (Sanger et al., Proc. Natl. Acad. Sci. USA 74:5463-5467 (1977)). Alternative methods have been and continue to be developed to increase the speed and ease of DNA sequencing. For example, high throughput and single molecule sequencing platforms are commercially available or under development from 454 Life Sciences (Branford, Conn.) (Margulies et al., Nature 437:376-380 (2005)); Solexa (Hayward, Calif.); Helicos BioSciences Corporation (Cambridge, Mass.) (U.S. application Ser. No. 11/167,046, filed Jun. 23, 2005), and Li-Cor Biosciences (Lincoln, Nebr.) (U.S. application Ser. No. 11/118,031, filed Apr. 29, 2005).

After an individual's genomic profile is generated, the profile is stored digitally. The profile may be stored digitally in a secure manner. The genomic profile is encoded in a computer readable format, such as on a computer readable medium, to be stored as part of a data set and may be stored as a database, where the genomic profile may be “banked”, and can be accessed again later. The data set comprises a plurality of data points, wherein each data point relates to an individual. Each data point may have a plurality of data elements. One data element is the unique identifier, used to identify the individual's genomic profile. The unique identifier may be a bar code. Another data element is genotype information, such as the SNPs or nucleotide sequence of the individual's genome. Data elements corresponding to the genotype information may also be included in the data point. For example, if the genotype information includes SNPs identified by microarray analysis, other data elements may include the microarray SNP identification number. Alternatively, if the genotype information was identified by other means, such as by RT-PCR methods (such as TaqMan assays), the data element may include level of fluorescence, primer information, and probe sequence. Other data elements may include, but not be limited to, SNP rs number, polymorphic nucleotide, chromosome position of the genotype information, quality metrics of the data, raw data files, images of the data, and extracted intensity scores.

The individual's specific factors such as physical data, medical data, ethnicity, ancestry, geography, gender, age, family history, known phenotypes, demographic data, exposure data, lifestyle data, behavior data, and other known phenotypes may also be incorporated as data elements. For example, factors may include, but are not limited to, an individual's birthplace, parents and/or grandparents, relatives' ancestry, location of residence, ancestors' location of residence, environmental conditions, known health conditions, known drug interactions, family health conditions, lifestyle conditions, diet, exercise habits, marital status, and physical measurements, such as weight, height, cholesterol level, heart rate, blood pressure, glucose level and other measurements known in the art The above mentioned factors for an individual's relatives or ancestors, such as parents and grandparents, may also be incorporated as data elements and used to determine an individual's risk for a phenotype or condition.

The specific factors may be obtained from a questionnaire or from a health care manager of the individual. Information from the “banked” profile can then be accessed and utilized as desired. For example, in the initial assessment of an individual's genotype correlations, the individual's entire information (typically SNPs or other genomic sequences across, or taken from an entire genome) will be analyzed for genotype correlations. In subsequent analyses, either the entire information can be accessed, or a portion thereof, from the stored, or banked genomic profile, as desired or appropriate.

### Correlations and Phenotype Profiles

The genomic profile is used to generate phenotype profiles. The genomic profile is typically stored digitally and is readily accessed at any point of time to generate phenotype profiles. Phenotype profiles are generated by applying rules that correlate or associate genotypes with phenotypes. Rules can be made based on scientific research that demonstrates a correlation between a genotype and a phenotype. The correlations may be curated or validated by a committee of one or more experts. By applying the rules to a genomic profile of an individual, the association between an individual's genotype and a phenotype may be determined. The phenotype profile for an individual will have this determination. The determination may be a positive association between an individual's genotype and a given phenotype, such that the individual has the given phenotype, or will develop the phenotype. Alternatively, it may be determined that the individual does not have, or will not develop, a given phenotype. In other embodiments, the determination may be a risk factor, estimate, or a probability that an individual has, or will develop a phenotype.

The determinations may be made based on a number of rules, for example, a plurality of rules may be applied to a genomic profile to determine the association of an individual's genotype with a specific phenotype. The determinations may also incorporate factors that are specific to an individual, such as ethnicity, gender, lifestyle (for example, diet and exercise habits), age, environment (for example, location of residence), family medical history, personal medical history, and other known phenotypes. The incorporation of the specific factors may be by modifying existing rules to encompass these factors. Alternatively, separate rules may be generated by these factors and applied to a phenotype determination for an individual after an existing rule has been applied.

Phenotypes may include any measurable trait or characteristic, such as susceptibility to a certain disease or response to a drug treatment. Other phenotypes that may be included are physical and mental traits, such as height, weight, hair color, eye color, sunburn susceptibility, size, memory, intelligence, level of optimism, and general disposition. Phenotypes may also include genetic comparisons to other individuals or organisms. For example, an individual may be interested in the similarity between their genomic profile and that of a celebrity. They may also have their genomic profile compared to other organisms such as bacteria, plants, or other animals. Together, the collection of correlated phenotypes determined for an individual comprises the phenotype profile for the individual.

Correlations between genetic variations and phenotypes can be obtained from scientific literature. Correlations for genetic variations are determined from analysis of a population of individuals who have been tested for the presence or absence of one or more phenotypic traits of interest and their genotype profile. The alleles of each genetic variation or polymorphism in the profile are reviewed to determine whether the presence or absence of a particular allele is associated with a trait of interest. Correlation can be performed by standard statistical methods and statistically significant correlations between genetic variations and phenotypic characteristics are noted. For example, it may be determined that the presence of allele A1 at polymorphism A correlates with heart disease. As a further example, it might be found that the combined presence of allele A1 at polymorphism A and allele B1 at polymorphism B correlates with increased risk of cancer. The results of the analyses may be published in peer-reviewed literature, validated by other research groups, and/or analyzed by a committee of experts, such as geneticists, statisticians, epidemiologists, and physicians, and may also be curated. For example, correlations disclosed in US Publication No. 20080131887 and PCT Publication No. WO/2008/067551 may be used in the embodiments described herein.

Alternatively, the correlations may be generated from the stored genomic profiles. For example, individuals with stored genomic profiles may also have known phenotype information stored as well. Analysis of the stored genomic profiles and known phenotypes may generate a genotype correlation. As an example, 250 individuals with stored genomic profiles also have stored information that they have previously been diagnosed with diabetes. Analysis of their genomic profiles is performed and compared to a control group of individuals without diabetes. It is then determined that the individuals previously diagnosed with diabetes have a higher rate of having a particular genetic variant compared to the control group, and a genotype correlation may be made between that particular genetic variant and diabetes.

Rules are made based on the validated correlations of genetic variants to particular phenotypes. Rules may be generated based on the genotypes and phenotypes correlated as disclosed in US Publication No. 20080131887 and PCT Publication No. WO/2008/067551, and some rules maybe incorporate other factors such as gender or ethnicity to generate effects estimates. Other measures resulting from rules may be estimated relative risk increase. The effects estimates and estimated relative risk increase may be from the published literature, or calculated from the published literature. Alternatively, the rules may be based on correlations generated from stored genomic profiles and previously known phenotypes.

Genetic variants may include SNPs. While SNPs occur at a single site, individuals who carry a particular SNP allele at one site often predictably carry specific SNP alleles at other sites. A correlation of SNPs and an allele predisposing an individual to disease or condition occurs through linkage disequilibrium, in which the non-random association of alleles at two or more loci occur more or less frequently in a population than would be expected from random formation through recombination.

Other genetic markers or variants, such as nucleotide repeats or insertions, may also be in linkage disequilibrium with genetic markers that have been shown to be associated with specific phenotypes. For example, a nucleotide insertion is correlated with a phenotype and a SNP is in linkage disequilibrium with the nucleotide insertion. A rule is made based on the correlation between the SNP and the phenotype. A rule based on the correlation between the nucleotide insertion and the phenotype may also be made. Either rules or both rules may be applied to a genomic profile, as the presence of one SNP may give a certain risk factor, the other may give another risk factor, and when combined may increase the risk.

Through linkage disequilibrium, a disease predisposing allele cosegregates with a particular allele of a SNP or a combination of particular alleles of SNPs. A particular combination of SNP alleles along a chromosome is termed a haplotype, and the DNA region in which they occur in combination can be referred to as a haplotype block. While a haplotype block can consist of one SNP, typically a haplotype block represents a contiguous series of 2 or more SNPs exhibiting low haplotype diversity across individuals and with generally low recombination frequencies. An identification of a haplotype can be made by identification of one or more SNPs that lie in a haplotype block. Thus, a SNP profile typically can be used to identify haplotype blocks without necessarily requiring identification of all SNPs in a given haplotype block.

Genotype correlations between SNP haplotype patterns and diseases, conditions or physical states are increasingly becoming known. For a given disease, the haplotype patterns of a group of people known to have the disease are compared to a group of people without the disease. By analyzing many individuals, frequencies of polymorphisms in a population can be determined, and in turn these frequencies or genotypes can be associated with a particular phenotype, such as a disease or a condition. Examples of known SNP-disease correlations include polymorphisms in Complement Factor H in age-related macular degeneration (Klein et al., Science: 308:385-389, (2005)) and a variant near the INSIG2 gene associated with obesity (Herbert et al., Science: 312:279-283 (2006)). Other known SNP correlations include polymorphisms in the 9p21 region that includes CDKN2A and B, such as ) such as rs10757274, rs2383206, rs13333040, rs2383207, and rs10116277 correlated to myocardial infarction (Helgadottir et al., Science 316:1491-1493 (2007); McPherson et al., Science 316:1488-1491 (2007))

The SNPs may be functional or non-functional. For example, a functional SNP has an effect on a cellular function, thereby resulting in a phenotype, whereas a non-functional SNP is silent in function, but may be in linkage disequilibrium with a functional SNP. The SNPs may also be synonymous or non-synonymous. SNPs that are synonymous are SNPs in which the different forms lead to the same polypeptide sequence, and are non-functional SNPs. If the SNPs lead to different polypetides, the SNP is non-synonymous and may or may not be functional. SNPs, or other genetic markers, used to identify haplotypes in a diplotype, which is 2 or more haplotypes, may also be used to correlate phenotypes associated with a diplotype. Information about an individual's haplotypes, diplotypes, and SNP profiles may be in the genomic profile of the individual.

Typically, for a rule to be generated based on a genetic marker in linkage disequilibrium with another genetic marker that is correlated with a phenotype, the genetic marker has a r2 or D′ score (scores commonly used in the art to determine linkage disequilibrium) of greater than 0.5. The score can be greater than approximately 0.5, 0.6, 0.7, 0.8, 0.90, 0.95 or 0.99. As a result, the genetic marker used to correlate a phenotype to an individual's genomic profile may be the same as the functional or published SNP correlated to a phenotype, or different. In some embodiments, the test SNP may not yet be identified, but using the published SNP information, allelic differences or SNPs may be identified based on another assay, such as TaqMan. For example, a published SNP is rs1061170 but a test SNP has not been identified. The test SNP may be identified by LD analysis with the published SNP. Alternatively, the test SNP may not be used, and instead, TaqMan or other comparable assay, will be used to assess an individual's genome having the test SNP.

The test SNPs may be “DIRECT” or “TAG” SNPs. Direct SNPs are the test SNPs that are the same as the published or functional SNP. For example, the direct SNP may be used for FGFR2 correlation with breast cancer, using the SNP rs1073640 in Europeans and Asians, where the minor allele is A and the other allele is G (Easton et al., Nature 447:1087-1093 (2007)). Another published or functional SNP that can be a direct SNP for FGFR2 correlation to breast cancer is rs1219648, also in Europeans and Asians (Hunter et al., Nat. Genet. 39:870-874 (2007)). Tag SNPs are where the test SNP is different from that of the functional or published SNP. Tag SNPs may also be used for other genetic variants such as SNPs for CAMTA1 (rs4908449), 9p21 (rs10757274, rs2383206, rs13333040, rs2383207, rs10116277), COL1A1 (rs1800012), FVL (rs6025), HLA-DQA1 (rs4988889, rs2588331), eNOS (rs1799983), MTHFR (rs1801133and APC (rs28933380).

Databases of SNPs are publicly available from, for example, the International HapMap Project (see www.hapmap.org, The International HapMap Consortium, Nature 426:789-796 (2003), and The International HapMap Consortium, Nature 437:1299-1320 (2005)), the Human Gene Mutation Database (HGMD) public database (see www.hgmd.org), and the Single Nucleotide Polymorphism database (dbSNP) (see www.ncbi.nlm.nih.gov/SNP/). These databases provide SNP haplotypes, or enable the determination of SNP haplotype patterns. Accordingly, these SNP databases enable examination of the genetic risk factors underlying a wide range of diseases and conditions, such as cancer, inflammatory diseases, cardiovascular diseases, neurodegenerative diseases, and infectious diseases. The diseases or conditions may be actionable, in which treatments and therapies currently exist. Treatments may include prophylactic treatments as well as treatments that ameliorate symptoms and conditions, including lifestyle changes.

Many other phenotypes such as physical traits, physiological traits, mental traits, emotional traits, ethnicity, ancestry, and age may also be examined. Physical traits may include height, hair color, eye color, body, or traits such as stamina, endurance, and agility. Mental traits may include intelligence, memory performance, or learning performance. Ethnicity and ancestry may include identification of ancestors or ethnicity, or where an individual's ancestors originated from. The age may be a determination of an individual's real age, or the age in which an individual's genetics places them in relation to the general population. For example, an individual's real age is 38 years of age, however their genetics may determine their memory capacity or physical well-being may be of the average 28 year old. Another age trait may be a projected longevity for an individual.

Other phenotypes may also include non-medical conditions, such as “fun” phenotypes. These phenotypes may include comparisons to well known individuals, such as foreign dignitaries, politicians, celebrities, inventors, athletes, musicians, artists, business people, and infamous individuals, such as convicts. Other “fun” phenotypes may include comparisons to other organisms, such as bacteria, insects, plants, or non-human animals. For example, an individual may be interested to see how their genomic profile compares to that of their pet dog, or to a former president.

The rules are applied to the stored genomic profile to generate a phenotype profile. For example, correlation data from published sources, or from stored genomic profiles can form the basis of rules or tests, to apply to an individual's genomic profile. The rules may encompass the information on test SNP and alleles, and the effect estimates, such as OR, or odds-ratio (95% confidence interval) or mean. The effects estimate may be a genotypic risk, such as the risk for homozygotes (homoz or RR), risk heterozygotes (heteroz or RN), and nonrisk homozygotes (homoz or NN). The effect estimate can also be carrier risk, which is RR or RN vs NN. The effect estimate may be based on the allele, such as an allelic risk, an example being R vs. N. There may also be 2, 3, 4, or more loci genotypic effect estimates (e.g. RRRR, RRNN, etc for the 9 possible genotype combinations for a two locus effect estimate).

The estimated risk for a condition may be based on the SNPs as listed in US Publication No. 20080131887 and PCT Publication No. WO/2008/067551. In some embodiments, the risk for a condition may be based on at least one SNP. For example, assessment of an individual's risk for Alzheimers (AD), colorectal cancer (CRC), osteoarthritis (OA) or exfoliation glaucoma (XFG), may be based on 1 SNP (for example, rs4420638 for AD, rs6983267 for CRC, rs4911178 for OA and rs2165241 for XFG). For other conditions, such as obesity (BMIOB), Graves' disease (GD), or hemochromatosis (HEM), an individual's estimated risk may be based on at least 1 or 2 SNPs (for example, rs9939609 and/or rs9291171 for BMIOB; DRB1*0301 DQA1*0501 and/or rs3087243 for GD; rs1800562 and/or rs129128 for HEM). For conditions such as, but not limited to, myocardial infarction (MI), multiple sclerosis (MS), or psoriasis (PS), 1, 2, or 3 SNPs may be used to assess an individual's risk for the condition (for example, rs1866389, rs1333049, and/or rs6922269 for MI; rs6897932, rs12722489, and/or DRB1*1501 for MS; rs6859018, rs11209026, and/or HLAC*0602 for PS). For estimating an individual's risk of restless legs syndrome (RLS) or celiac disease (CelD), 1, 2, 3, or 4 SNPs (for example, rs6904723, rs2300478, rs1026732, and/or rs9296249 for RLS; rs6840978, rs11571315, rs2187668, and/or DQA1*0301 DQB1*0302 for CelD). For prostate cancer (PC) or lupus (SLE), 1, 2, 3, 4, or 5 SNPs may be used to estimate an individual's risk for PC or SLE (for example, rs4242384, rs6983267, rs16901979, rs17765344, and/or rs4430796 for PC; rs12531711, rs10954213, rs2004640, DRB1*0301, and/or DRB1*1501 for SLE). For estimating an individual's lifetime risk of macular degeneration (AMD) or rheumatoid arthritis (RA), 1, 2, 3, 4, 5, or 6 SNPs, may be used (for example, rs10737680, rs10490924, rs541862, rs2230199, rs1061170, and/or rs9332739 for AMD; rs6679677, rs11203367, rs6457617, DRB*0101, DRB1*0401, and/or DRB1*0404 for RA). For estimating an individual's lifetime risk of breast cancer (BC), 1, 2, 3, 4, 5, 6 or 7 SNPs may be used (for example, rs3803662, rs2981582, rs4700485, rs3817198, rs17468277, rs6721996, and/or rs3803662). For estimating an individual's lifetime risk of Crohn's disease (CD) or Type 2 diabetes (T2D), 1, 2, 3, 4, 5, 6, 7, 8, 9, 10, or 11 SNPs may be used (for example, rs2066845, rs5743293, rs10883365, rs17234657, rs10210302, rs9858542, rs1805303, rs1000113,rs17221417, rs2542151, and/or rs10761659 for CD; rs13266634, rs4506565, rs10012946, rs7756992, rs10811661, rs12288738, rs8050136, rs1111875, rs4402960, rs5215, and/or rs1801282 for T2D). In some embodiments, the SNPs used as a basis for determining risk may be in linkage disequilibrium with the SNPs as mentioned above, or other SNPs, such as in US Publication No. 20080131887 and PCT Publication No. WO/2008/067551.

The phenotype profile of an individual may comprise a number of phenotypes. In particular, the assessment of a patient's risk of disease or other conditions such as likely drug response including metabolism, efficacy and/or safety, by the methods disclosed herein, allows for prognostic or diagnostic analysis of susceptibility to multiple, unrelated diseases and conditions, whether in symptomatic, presymptomatic or asymptomatic individuals, including carriers of one or more disease/condition predisposing alleles. Accordingly, these methods provide for general assessment of an individual's susceptibility to disease or condition without any preconceived notion of testing for a specific disease or condition. For example, the methods disclosed herein allow for assessment of an individual's susceptibility to any of the several conditions listed in US Publication No. 20080131887 and PCT Publication No. WO/2008/067551, based on the individual's genomic profile. Furthermore, the methods allow assessments of an individual's estimated lifetime risk or relative risk for one or more phenotype or condition.

The assessment provides information for 2 or more of these conditions, and can include at least 3, 4, 5, 10, 15, 18, 20, 25, 30, 35, 40, 45, 50, 100 or even more of these conditions. A single rule for a phenotype may be applied for monogenic phenotypes. More than one rule may also be applied for a single phenotype, such as a multigenic phenotype or a monogenic phenotype wherein multiple genetic variants within a single gene affects the probability of having the phenotype.

Following an initial screening of an individual patient's genomic profile, updates of an individual's genotype correlations can be made (or are available) through comparisons to additional genetic variants, such as SNPs, when such additional genetic variants become known. For example, updates may be performed periodically, for example, daily, weekly, or monthly by one or more people of ordinary skill in the field of genetics, who scan scientific literature for new genotype correlations. The new genotype correlations may then be further validated by a committee of one or more experts in the field.

The new rule may encompass a genotype or phenotype without an existing rule. For example, a genotype not correlated with any phenotype is discovered to correlate with a new or existing phenotype. A new rule may also be for a correlation between a phenotype for which no genotype has previously been correlated to. New rules may also be determined for genotypes and phenotypes that have existing rules. For example, a rule based on the correlation between genotype A and phenotype A exists. New research reveals genotype B correlates with phenotype A, and a new rule based on this correlation is made. Another example is phenotype B is discovered to be associated with genotype A, and thus a new rule may be made.

Rules may also be made on discoveries based on known correlations but not initially identified in published scientific literature. For example, it may be reported genotype C is correlated with phenotype C. Another publication reports genotype D is correlated with phenotype D. Phenotype C and D are related symptoms, for example phenotype C may be shortness of breath, and phenotype D is small lung capacity. A correlation between genotype C and phenotype D, or genotype D with phenotype C, may be discovered and validated through statistical means with existing stored genomic profiles of individuals with genotypes C and D, and phenotypes C and D, or by further research. A new rule may then be generated based on the newly discovered and validated correlation. In another embodiment, stored genomic profiles of a number of individuals with a specific or related phenotype may be studied to determine a genotype common to the individuals, and a correlation may be determined. A new rule may be generated based on this correlation.

Rules may also be made to modify existing rules. For example, correlations between genotypes and phenotypes may be partly determined by a known individual characteristic, such as ethnicity, ancestry, geography, gender, age, family history, or any other known phenotypes of the individual. Rules based on these known individual characteristics may be made and incorporated into an existing rule, to provide a modified rule. The choice of modified rule to be applied will be dependent on the specific individual factor of an individual. For example, a rule may be based on the probability an individual who has phenotype E is 35% when the individual has genotype E. However, if an individual is of a particular ethnicity, the probability is 5%. A new rule may be generated based on this result and applied to individuals with that particular ethnicity. Alternatively, the existing rule with a determination of 35% may be applied, and then another rule based on ethnicity for that phenotype is applied. The rules based on known individual characteristics may be determined from scientific literature or determined based on studies of stored genomic profiles. New rules may be added and applied to genomic profiles, as the new rules are developed, or they may be applied periodically, such as at least once a year.

Information of an individual's risk of disease can also be expanded as technology advances allow for finer resolution SNP genomic profiles. As indicated above, an initial SNP genomic profile readily can be generated using microarray technology for scanning of 500,000 SNPs. Given the nature of haplotype blocks, this number allows for a representative profile of all SNPs in an individual's genome. Nonetheless, there are approximately 10 million SNPs estimated to occur commonly in the human genome (the International HapMap Project; www.hapmap.org). As technological advances allow for practical, cost-efficient resolution of SNPs at a finer level of detail, such as microarrays of 1,000,000, 1,500,000, 2,000,000, 3,000,000, or more SNPs, or whole genomic sequencing, more detailed SNP genomic profiles can be generated. Likewise, cost-efficient analysis of finer SNP genomic profiles and updates to the master database of SNP-disease correlations will be enabled by advances in computational analytical methodology.

In some embodiments, “field-deployed” mechanisms may be gathered from individuals, and incorporated into the phenotype profile for the individuals. For example, an individual may have an initial phenotype profile generated based on genetic information. The initial phenotype profile generated includes risk factors for different phenotypes as well as suggested treatments or preventative measures, reported in a personal action plan. The profile may include information on available medication for a certain condition, and/or suggestions on dietary changes or exercise regimens. The individual may choose to see, or contact via a web portal or phone call, a physician or genetic counselor, to discuss their phenotype profile. The individual may decide to take a certain course of action, for example, take specific medications, change their diet, and other possible actions suggested on their personal action plan. The individual may then subsequently submit biological samples to assess changes in their physical condition and possible change in risk factors.

Individuals may have the changes determined by directly submitting biological samples to the facility (or associated facility, such as a facility contracted by the entity generating the genetic profiles and phenotype profiles) that generates the genomic profiles and phenotype profiles. Alternatively, the individuals may use a “field-deployed” mechanism, wherein the individual may submit their saliva, blood, or other biological sample into a detection device at their home, analyzed by a third party, and the data transmitted to be incorporated into another phenotype profile. For example, an individual may have received an initial phenotype report based on their genetic data reporting the individual having an increased lifetime risk of myocardial infarction (MI). The report may also have suggestions on preventative measures to reduce the risk of MI, such as cholesterol lowering drugs and change in diet. The individual may choose to contact a genetic counselor or physician to discuss the report and the preventative measures and decides to change their diet. After a period of being on the new diet, the individual may see their personal physician to have their cholesterol level measured. The new information (cholesterol level) may be transmitted (for example, via the Internet) to the entity with the genomic information, and the new information used to generate a new phenotype profile for the individual, with a new risk factor for myocardial infarction, and/or other conditions.

The individual may also use a “field-deployed” mechanism, or direct mechanism, to determine their individual response to specific medications. For example, an individual may have their response to a drug measured, and the information may be used to determine more effective treatments. Measurable information include, but are not limited to, metabolite levels, glucose levels, ion levels (for example, calcium, sodium, potassium, iron), vitamins, blood cell counts, body mass index (BMI), protein levels, transcript levels, heart rate, etc., can be determined by methods readily available and can be factored into an algorithm to combine with initial genomic profiles to determine a modified overall risk estimate score. The risk estimate score may be a GCI score.

### Genetic Composite Index (GCI)

In some embodiments, information about the association of multiple genetic markers or variants with one or more diseases or conditions is combined and analyzed to produce a Genetic Composite Index (GCI) score. This score incorporates known risk factors, as well as other information and assumptions such as the allele frequencies and the prevalence of a disease. The GCI can be used to qualitatively estimate the association of a disease or a condition with the combined effect of a set of genetic markers. The GCI score can be used to provide people not trained in genetics with a reliable (i.e., robust), understandable, and/or intuitive sense of what their individual risk of a disease is compared to a relevant population based on current scientific research.

The GCI score may be used to generate GCI Plus scores. The methods disclosed herein encompasses using the GCI score, and one of ordinary skill in the art will readily recognize the use of GCI Plus scores or variations thereof, in place of GCI scores as described herein. The GCI Plus score may contain all the GCI assumptions, including risk (such as lifetime risk), age-defined prevalence, and/or age-defined incidence of the condition. The lifetime risk for the individual may then be calculated as a GCI Plus score which is proportional to the individual's GCI score divided by the average GCI score. The average GCI score may be determined from a group of individuals of similar ancestral background, for example a group of Caucasians, Asians, East Indians, or other group with a common ancestral background. Groups may comprise of at least 5, 10, 15, 20, 25, 30, 35, 40, 45, 50, 55, or 60 individuals. In some embodiments, the average may be determined from at least 75, 80, 95, or 100 individuals. The GCI Plus score may be determined by determining the GCI score for an individual, dividing the GCI score by the average relative risk and multiplying by the lifetime risk for a condition or phenotype. For example, using data from US Publication No. 20080131887 and PCT Publication No. WO/2008/067551, GCI or GCI Plus scores for an individual can be determined. The scores may be used to generate information on genetic risks, such as estimated lifetime risk, for one or more conditions in the phenotype profile of an individual. The methods allow calculating estimated lifetime risks or relative risks for one or more phenotypes or conditions. The risk for a single condition may be based on one or more SNP. For example, an estimated risk for a phenotype or condition may be based on at least 2, 3, 4, 5, 6, 7, 8, 9, 10, 11, or 12 SNPs, wherein the SNPs for estimating a risk may be published SNPs, test SNPs, or both.

A GCI score can be generated for each disease or condition of interest. These GCI scores may be collected to form a risk profile for an individual. The GCI scores may be stored digitally so that they are readily accessible at any point of time to generate risk profiles. Risk profiles may be broken down by broad disease classes, such as cancer, heart disease, metabolic disorders, psychiatric disorders, bone disease, or age on-set disorders. Broad disease classes may be further broken down into subcategories. For example for a broad class such as a cancer, sub-categories of cancer may be listed such as by type (sarcoma, carcinoma or leukemia, etc.) or by tissue specificity (neural, breast, ovaries, testes, prostate, bone, lymph nodes, pancreas, esophagus, stomach, liver, brain, lung, kidneys, etc.). Further the risk profiles may display information on how the GCI scores are predicted to change as the individual ages or various risk factors are adjusted. For example, the GCI scores for particular diseases may take into account the effect of changes in diet or preventative measures taken (smoking cessation, drug intake, double radical mastectomies, hysterectomies, and the like).

A GCI score can be generated for an individual, which provides them with easily comprehended information about the individual's risk of acquiring or susceptibility to at least one disease or condition. One or more GCI scores can be generated for a single disease or condition, or numerous diseases or conditions. The one or more GCI score can be accessible by an on-line portal. Alternatively, the one or more GCI scores may be provided in paper form, with subsequent updates also provided in paper form. The paper form can be mailed to an individual or their health care manager or provided in person.

A method for generating a robust GCI score for the combined effect of different loci can be based on a reported individual risk for each locus studied. For example, a disease or condition of interest is identified and then informational sources, including but not limited to databases, patent publications and scientific literature, are queried for information on the association of the disease of condition with one or more genetic loci. These informational sources are curated and assessed using quality criteria. In some embodiments the assessment process involves multiple steps. In other embodiments the informational sources are assessed for multiple quality criteria. The information derived from informational sources is used to identify the odds ratio or relative risk for one or more genetic loci for each disease or condition of interest.

In an alternative embodiment, the odds ratio (OR) or relative risk (RR) for at least one genetic loci is not available or not accessible from informational sources. The RR is then calculated using (1) reported OR of multiple alleles of the same locus, (2) allele frequencies from data sets, such as the HapMap data set, and/or (3) disease/condition prevalence from available sources (e.g., CDC, National Center for Health Statistics, etc.) to derive RR of all alleles of interest. In one embodiment the ORs of multiple alleles of same locus are estimated separately or independently. In a preferred embodiment the ORs of multiple alleles of same locus are combined to account for dependencies between the ORs of the different alleles. In some embodiments established disease models (including, but not limited to models such as the multiplicative, additive, Harvard-modified, dominant effect) are used to generate an intermediate score that represents the risk of an individual according to the model chosen.

A method that can be used analyzes multiple models for a disease or condition of interest and correlates the results obtained from these different models; this minimizes the possible errors that may be introduced by choice of a particular disease model. This method minimizes the influence of reasonable errors in the estimates of prevalence, allele frequencies and ORs obtained from informational sources on the calculation of the relative risk. Without being limited by theory, because of the “linearity” or monotonic nature of the effect of a prevalence estimate on the RR, there is little or no effect of incorrectly estimating the prevalence on the final rank score; provided that the same model is applied consistently to all individuals for which a report is generated.

The methods described herein can also take into account environmental/behavioral/demographic data as additional “loci.” In a related method, such data may be obtained from informational sources, such as medical or scientific literature or databases (e.g., associations of smoking w/lung cancer, or from insurance industry health risk assessments). Also disclosed herein are GCI scores produced for one or more complex diseases. Complex diseases may be influenced by multiple genes, environmental factors, and their interactions. A large number of possible interactions may need to be analyzed when studying complex diseases. A procedure used to correct for multiple comparisons, such as the Bonferroni correction, may be used to generate a GCI score. Alternatively, the Simes's test can be used to control the overall significance level (also known as the “familywise error rate”) when the tests are independent or exhibit a special type of dependence (Sarkar S., Ann Stat 26:494-504 (1998)). Simes's test rejects the global null hypothesis that all K test-specific null hypotheses are true if p₍ₖ₎≦αk/K for any k in 1, . . . , K. (Simes, R. J., Biometrika 73:751-754 (1986)).

Other embodiments that can be used in the context of multiple-gene and multiple-environmental-factor analysis control the false-discovery rate—that is, the expected proportion of rejected null hypotheses that are falsely rejected. This approach can be particularly useful when a portion of the null hypotheses can be assumed false, as in microarray studies. Devlin et al. (Genet. Epidemiol. 25:36-47 (2003)) proposed a variant of the Benjamini and Hochberg (J. R. Stat. Soc. Ser. B 57:289-300 (1995)) step-up procedure that controls the false-discovery rate when testing a large number of possible gene×gene interactions in multilocus association studies. The Benjamini and Hochberg procedure is related to Simes's test; setting k*=maxk such that p₍ₖ₎≦αk/K, it rejects all k* null hypotheses corresponding to p₍₁₎, . . . , p₍ₖ₎*. In fact, the Benjamini and Hochberg procedure reduces to Simes's test when all null hypotheses are true (Benjamini and Yekutieli, Ann. Stat. 29:1165-1188 (2001)).

Also provided herein is a ranking of an individual, where an individual is ranked in comparison to a population of individuals based on their intermediate score to produce a final rank score, which may be represented as rank in the population, such as the 99^{th} percentile or 99^{th}, 98^{th}, 97^{th}, 96^{th}, 95^{th}, 94^{th}, 93^{rd}, 92^{nd}, 91^{st}, 90^{th}, 89^{th}, 88^{th}, 87^{th}, 86^{th}, 85^{th}, 84^{th}, 83^{rd}, 82^{nd}, 81^{st}, 80^{th}, 79^{th}, 78^{th}, 77^{th}, 76^{th}, 75^{th}, 74^{th}, 73^{rd}, 72^{nd}, 71^{st}, 70^{th}, 69^{th}, 65^{th}, 60^{th}, 55^{th}, 50^{th}, 45^{th}, 40^{th}, 40^{th}, 35^{th}, 30^{th}, 25^{th}, 20^{th}, 15^{th}, 10^{th}, 5^{th}, or 0^{th} percentile. The rank score may be displayed as a range, such as the 100^{th} to 95^{th} percentile, the 95^{th} to 85^{th} percentile, the 85^{th} to 60^{th} percentile, or any sub-range between the 100^{th} and 0th percen individual can also be ranked in quartiles, such as the top 75^{th} quartile, or the lowest 25^{th} quartile. The individual can also be ranked in comparison to the mean or median score of the population.

In one embodiment, the population to which the individual is compared to includes a large number of people from various geographic and ethnic backgrounds, such as a global population. Alternatively, the population to which an individual is compared to is limited to a particular geography, ancestry, ethnicity, sex, age (for example, fetal, neonate, child, adolescent, teenager, adult, geriatric), or disease state (for example, symptomatic, asymptomatic, carrier, early-onset, late onset). In some embodiments, the population to which the individual is compared to is derived from information reported in public and/or private informational sources.

The GCI score can be generated using a multi-step process. For example, initially, for each condition to be studied, the relative risks from the odds ratios for each of the genetic markers is calculated. For every prevalence value p=0.01,0.02, . . . ,0.5, the GCI score of the HapMap CEU population is calculated based on the prevalence and on the HapMap allele frequency. If the GCI scores are invariant under the varying prevalence, then the only assumption taken into account is that there is a multiplicative model. Otherwise, it is determined that the model is sensitive to the prevalence. The relative risks and the distribution of the scores in the HapMap population, for any combination of no-call values, are obtained. For each new individual, the individual's score is compared to the HapMap distribution and the resulting score is the individual's rank in this population. The resolution of the reported score may be low due to the assumptions made during the process. The population will be partitioned into quantiles (3-6 bins), and the reported bin would be the one in which the individual's rank falls. The number of bins may be different for different diseases based on considerations such as the resolution of the score for each disease. In case of ties between the scores of different HapMap individuals, the average rank will be used.

A higher GCI score can be interpreted as an indication of an increased risk for acquiring or being diagnosed with a condition or disease. Mathematical models are typically used to derive the GCI score. The GCI score can be based on a mathematical model that accounts for the incomplete nature of the underlying information about the population and/or diseases or conditions. The mathematical model can include at least one presumption as part of the basis for calculating the GCI score, wherein the presumption includes, but is not limited to: a presumption that the odds ratio values are given; a presumption that the prevalence of the condition is known; a presumption that the genotype frequencies in the population are known; and/or a presumption that the customers are from the same ancestry background as the populations used for the studies and as the HapMap; a presumption that the amalgamated risk is a product of the different risk factors of the individual genetic markers. The GCI may also include a presumption that the multi-genotypic frequence of a genotype is the product of frequencies of the alleles of each of the SNPs or individual genetic markers (for example, the different SNPs or genetic markers are independent across the population).

The Multiplicative Model

The GCI score can be computed under the assumption that the risk attributed to the set of genetic markers is the product of the risks attributed to the individual genetic markers. Thus, the different genetic markers attribute independently of the other genetic markers to the risk of the disease. Formally, there are k genetic markers with risk alleles r₁, . . . ,rₖ and non-risk alleles n₁, . . . ,nₖ. In SNP i, the three possible genotype values are denoted as rᵢrᵢ,nᵢrᵢ, and nᵢnᵢ. The genotype information of an individual can be described by a vector, (g₁, . . . ,gₖ), where gᵢ can be 0,1, or 2, according to the number of risk alleles in position i. Denoted by λ₁ⁱ, the relative risk of a heterozygous genotype in position i compared to a homozygous non-risk allele at the same position. In other words,

$$λ \frac{i}{1} = \frac{P ( D \vert {n}_{i} {r}_{i} \vert )}{P ( D \vert {n}_{i} {n}_{i} \vert )} .$$

Similarly, the relative risk of an rᵢrᵢ genotype is denoted as

$$λ \frac{i}{2} = \frac{P ( D \vert {n}_{i} {r}_{i} \vert )}{P ( D \vert {n}_{i} {n}_{i} \vert )} .$$

Under the multiplicative model, the assumption that the risk of an individual with a genotype (g₁, . . . ,gₖ) is

$$GCI ( {g}_{1} , … , {g}_{k} ) = {\prod }_{i = 1}^{k} {λ}_{{g}_{i}}^{i} .$$

Estimating the Relative Risk.

In another embodiment, the relative risks for different genetic markers are known and the multiplicative model can be used for risk assessment. However, in some embodiments involving association studies, the study design prevents the reporting of the relative risks. In some case-control studies the relative risk cannot be calculated directly from the data without further assumptions. Instead of reporting the relative risks, it is customary to report the odds ratio (OR) of the genotype, which are the odds of carrying the disease given the risk genotype (either rᵢrᵢ or nᵢrᵢ) vs. the odds of not carrying the disease given the risk genotypes. Formally,

$${OR}_{i}^{1} = \frac{P ( D \vert {n}_{i} {r}_{i} \vert )}{P ( D \vert {n}_{i} {r}_{i} \vert )} \cdot \frac{1 - P ( D \vert {n}_{i} {n}_{i} \vert )}{1 - P ( D \vert {n}_{i} {r}_{i} \vert )}$$

$${OR}_{i}^{2} = \frac{P ( D \vert {r}_{i} {r}_{i} \vert )}{P ( D \vert {n}_{i} {n}_{i} \vert )} \cdot \frac{1 - P ( D \vert {n}_{i} {n}_{i} \vert )}{1 - P ( D \vert {r}_{i} {r}_{i} \vert )}$$

Finding the relative risks from the odds ratio may require additional assumptions. Such as the presumption that the allele frequencies in an entire population

$$a = {f}_{{n}_{i} {n}_{i}} , b = {f}_{{n}_{i} {r}_{i}} , and c = {f}_{{r}_{i} {r}_{i}}$$

are known or estimated (these could be estimated from current datasets such as the HapMap dataset which includes 120 chromosomes), and/or that the prevalence of the disease p=p(D) is known. From the preceding three equations can be derived:

$$p = a \cdot P ( D \vert {n}_{i} {n}_{i} ) + b \cdot P ( D \vert {n}_{i} {r}_{i} ) + c \cdot P ( D \vert {r}_{i} {r}_{i} )$$

$${OR}_{i}^{1} = \frac{P ( D \vert {n}_{i} {r}_{i} \vert )}{P ( D \vert {n}_{i} {r}_{i} \vert )} \cdot \frac{1 - P ( D \vert {n}_{i} {n}_{i} \vert )}{1 - P ( D \vert {n}_{i} {r}_{i} \vert )} \\ {OR}_{i}^{2} = \frac{P ( D \vert {r}_{i} {r}_{i} \vert )}{P ( D \vert {n}_{i} {n}_{i} \vert )} \cdot \frac{1 - P ( D \vert {n}_{i} {n}_{i} \vert )}{1 - P ( D \vert {r}_{i} {r}_{i} \vert )}$$

By the definition of the relative risk, after dividing by the term pP(D|nᵢnᵢ), the first equation can be rewritten as:

$$\frac{1}{P ( D \vert {n}_{i} {n}_{i} )} = \frac{a + b {λ}_{1}^{i} + c {λ}_{2}^{i}}{p} ,$$

and therefore, the last two equations can be rewritten as:

$${OR}_{i}^{1} = {λ}_{1}^{i} \cdot \frac{( a - p ) + b {λ}_{1}^{i} + c {λ}_{2}^{i}}{a + ( b - p ) {λ}_{1}^{i} + c {λ}_{2}^{i}} \\ {OR}_{i}^{2} = {λ}_{2}^{i} \cdot \frac{( a - p ) + b {λ}_{1}^{i} + c {λ}_{2}^{i}}{a + b {λ}_{1}^{i} + ( c - p ) {λ}_{2}^{i}} \qquad ( 1 )$$

Note that when a=1 (non-risk allele frequency is 1), Equation system 1 is equivalent to the Zhang and Yu formula in Zhang and Yu (JAMA, 280:1690-1691 (1998)), which is incorporated by reference in its entirety. In contrast to the Zhang and Yu formula, some embodiments take into consideration the allele frequency in the population, which may affect the relative risk. Further, some embodiments take into account the interdependence of the relative risks, as opposed to computing each of the relative risks independently.

Equation system 1 can be rewritten as two quadratic equations, with at most four possible solutions. A gradient descent algorithm can be used to solve these equations, where the starting point is set to be the odds ratio, e.g., λ₁ⁱ=OR₁ⁱ, and λ₂ⁱ=OR₂ⁱ

For example:

f₁(λ₁,λ₂)=ORᵢ¹(a+(b−p)λ₁ⁱ+cλ₂ⁱ)−λ₁ⁱ·((a−p)+bλ₁ⁱ+cλ₂ⁱ)

f₂(λ₁,λ₂)=ORᵢ²(a+bλ₁ⁱ+(c−p)λ₂ⁱ)−λ₂ⁱ·((a−p)+bλ₁ⁱ+cλ₂ⁱ)

Finding the solution of these equations is equivalent to finding the minimum of the function g(λ₁,λ₂)=f₁(λ₁,λ₂)²+f₂(λ₁,λ₂)².

### Thus,

$$\frac{d g}{d {λ}_{1}} = 2 {f}_{1} ( {λ}_{1} , {λ}_{2} ) \cdot b \cdot ( {λ}_{2} - {OR}_{2} ) + 2 {f}_{2} ( {λ}_{1} , {λ}_{2} ) ( \begin{matrix}2 b {λ}_{1} + c {λ}_{2} + \\ a - {OR}_{1} b - \\ p + {OR}_{1} p\end{matrix} )$$

$$\frac{d g}{d {λ}_{2}} = 2 {f}_{2} ( {λ}_{1} , {λ}_{2} ) \cdot c \cdot ( {λ}_{1} - {OR}_{1} ) + 2 {f}_{1} ( {λ}_{1} , {λ}_{2} ) ( \begin{matrix}\begin{matrix}2 c {λ}_{2} + b {λ}_{1} + \\ a - {OR}_{2} c -\end{matrix} \\ p + {OR}_{2} p\end{matrix} )$$

In this example, by setting x₀=OR₁,y₀=OR₂. set the values [epsilon]=10⁻¹⁰ to be a tolerance the algorithm. In iteration i, define

$$γ = min \{ \begin{matrix}\begin{matrix}0.001 , \\ \frac{{x}_{i - 1}}{[ epsilon ] + 10 \vert \frac{d g}{d {λ}_{1}} ( {x}_{i - 1} , {y}_{i - 1} ) \vert} ,\end{matrix} \\ \frac{{y}_{i - 1}}{[ epsilon ] + 10 \vert \frac{d g}{d {λ}_{2}} ( {x}_{i - 1} , {y}_{i - 1} ) \vert}\end{matrix} \} , \\ then set$$

$${x}_{i} = {x}_{i - 1} - γ \frac{d g}{d {λ}_{1}} ( {x}_{i - 1} , {y}_{i - 1} )$$

$${y}_{i} = {y}_{i - 1} - γ \frac{d g}{d {λ}_{2}} ( {x}_{i - 1} , {y}_{i - 1} )$$

The iterations are repeated until g(xᵢ,yᵢ)<tolerance, where tolerance is set to 10⁻⁷ in the supplied code.

In this example, these equations give the correct solution for different values of a,b,c,p,OR₁ and OR².

Robustness of the Relative Risk Estimation.

In some embodiments, the effect of different parameters (prevalence, allele frequencies, and odds ratio errors) on the estimates of the relative risks is measured. In order to measure the effect of the allele frequency and prevalence estimates on the relative risk values, the relative risk from a set of values of different odds ratios and different allele frequencies is computed (under HWE), and the results of these calculations is plotted for prevalence values ranging from 0 to 1. Additionally, for fixed values of the prevalence, the resulting relative risks can be plotted as a function of the risk-allele frequencies. In cases when p=0, λ₁=OR₁, and λ₂=OR₂, and when p=1, λ₁=λ₂=0. This can be computed directly from the equations. Additionally, in some embodiments when the risk allele frequency is high, λ₁ gets closer to a linear function, and λ₂ gets closer to a concave function with a bounded second derivative. In the limit, when c=1,

$${λ}_{2} = {OR}_{2} + p ( 1 - {OR}_{2} ) , and {λ}_{i} = {OR}_{i} - \frac{( {OR}_{i} - 1 ) {pOR}_{i}}{{OR}_{2} ( 1 - p ) + {pOR}_{1}} .$$

If OR₁≈OR₂ the latter is close to a linear function as well. When risk-allele frequency is low, λ₁ and λ₂ approach the behavior of the function 1/p. In the limit, when c=0,

$${λ}_{1} = \frac{{OR}_{1}}{1 - p + {pOR}_{1}} , {λ}_{2} = \frac{{OR}_{2}}{1 - p + {pOR}_{2}} .$$

This indicates that for high risk-allele frequencies, incorrect estimates of the prevalence will not significantly affect the resulting relative risk. Further, for low risk-allele frequency, if a prevalence value of p′=αp is substituted for the correct prevalence p, then the resulting relative risks will be off by a factor of 1/α at most.

Calculating the GCI Score

In one embodiment, the GCI is calculated by using a reference set that represents the relevant population. This reference set may be one of the populations in the HapMap, or anther genotype dataset.

In this embodiment, the GCI is computed as follows: For each of the k risk loci, the relative risk is calculated from the odds ratio using the equation system 1. Then, the multiplicative score for each individual in the reference set is calculated. The GCI of an individual with a multiplicative score of s is the fraction of all individuals in the reference dataset with a score of s′≦s. For instance, if 50% of the individuals in the reference set have a multiplicative score smaller than s, the final GCI score of the individual would be 0.5. The GCI can be generalized to account for SNP-SNP interactions if the odds ratios or relative risks are known for the different genotype or haplotype combinations (these can be found in the literature in some cases).

As described herein, the multiplicative model can be used to in the GCI score, however, other models may be used for the purpose of determining the GCI score. Other suitable models include but are not limited to:

The Additive Model. Under the additive model, the risk of an individual with a genotype (g₁, . . . ,gₖ) is presumed to be

$$GCI ( {g}_{1} , … , {g}_{k} ) = {\sum }_{i = 1}^{k} {λ}_{{g}_{i}}^{i} .$$

Generalized Additive Model. Under the generalized additive model, it is presumed that there is a function f such that the risk of an individual with a genotype (g₁, . . . ,gₖ) is

$$GCI ( {g}_{1} , … , {g}_{k} ) = {\sum }_{i = 1}^{k} f ( {λ}_{{g}_{i}}^{i} ) .$$

Harvard Modified Score (Het). This score was derived from Colditz et al. (Cancer Causes and Controls, 11:477-488 (2000)), which is herein incorporated in its entirety. The Het score is essentially a generalized additive score, although the function f operates on the odds ratio values instead of the relative risks. This may be useful in cases where the relative risk is difficult to estimate. In order to define the function f, an intermediate function g, is defined as:

$$g ( x ) = \{ \begin{matrix}01 < x \leq 1.09 \\ 51.09 < x \leq 1.49 \\ 101.49 < x \leq 2.99 \\ 252.99 < x \leq 6.99 \\ 506.99 < x\end{matrix}$$

Next the quantity

$$het = {\sum }_{i = 1}^{k} {p}_{het}^{i} g ( O {R}_{1}^{i} )$$

is calculated, where pₕₑₜⁱ is the frequency of heterozygous individuals in SNP i across the reference population. The function f is then defined as f(x)=g(x)/het, and the Harvard Modified Score (Het) is simply defined as

$${\sum }_{i = 1}^{k} f ( O {R}_{{g}_{i}}^{i} ) .$$

The Harvard Modified Score (Hom). This score is similar to the Het score, except that the value het is replaced by the value

$$hom = {\sum }_{i = 1}^{k} {p}_{hom}^{i} g ( O {R}_{1}^{i} ) ,$$

where pₕₒₘⁱ is the frequency of individuals with homozygous risk-allele.

The Maximum-Odds Ratio. In this model, it is presumed that one of the genetic markers (one with a maximal odds ratio) gives a lower bound on the combined risk of the entire panel. Formally, the score of an individual with genotypes (g₁, . . . ,gₖ) is GCI(g₁, . . . ,gₖ)=maxᵢ₌₁^{k}OR_{g}_{ᵢ}ⁱ.

A comparison between the scores is described in Example 1 and GCI score evaluation is described in Example 2.

Extending the Model to an Arbitrary Number of Variants

The model can be extended to the situations where an arbitrary number of possible variants occur. Previous considerations dealt with situations where there were three possible variants (nn,nr,rr). Generally, when a multi-SNP association is known, an arbitrary number of variants may be found in the population. For example, when an interaction between two Genetic markers is associated with a condition, there are nine possible variants. This results in eight different odds ratios values.

To generalize the initial formula, it may be assumed that there are k+1 possible variants a₀, . . . ,aₖ, with frequencies f₀,f₁,. . . ,fₖ, measured odds ratios of 1,OR₁, . . . ,ORₖ, and unknown relative risk values 1,λ₁, . . . ,λₖ. Further it may be assumed that all relative risks and odds ratios are measured with respect to a₀, and thus,

$${λ}_{i} = \frac{P ( D \vert {a}_{i} )}{P ( D \vert {a}_{o} )} , and O {R}_{i} = \frac{P ( D \vert {a}_{i} )}{P ( D \vert {a}_{o} )} \cdot \frac{1 - P ( D \vert {a}_{i} )}{1 - P ( D \vert {a}_{o} )} .$$

### Based on:

$$p = {\sum }_{i = 1}^{k} {f}_{i} P ( D \vert {a}_{i} ) ,$$

It is determined that

$$O {R}_{i} = {λ}_{i} \frac{{\sum }_{i = 0}^{k} {f}_{i} {λ}_{i} - p}{{\sum }_{i = 0}^{k} {f}_{i} {λ}_{i} - {λ}_{i} p} .$$

Further if it is set that

$$C = {\sum }_{i} {f}_{i} {λ}_{i} ,$$

this results in the equation:

$${λ}_{i} = \frac{C \cdot O {R}_{i}}{C - p + O {R}_{i} p} , \\ and thus , \\ C = {\sum }_{i = 0}^{k} {f}_{i} {λ}_{i} = {\sum }_{i = 0}^{k} \frac{C \cdot O {R}_{i} {f}_{i}}{C - p + O {R}_{i} p} , \\ or$$

$$1 = {\sum }_{i = 0}^{k} \frac{O {R}_{i} {f}_{i}}{C - p + O {R}_{i} p} .$$

The latter is an equation with one variable (C). This equation can produce many different solutions (essentially, up to k+1 different solutions). Standard optimization tools such as gradient descent can be used to find the closest solution to C₀=Σfᵢtᵢ.

A robust scoring framework for the quantification of risk factors us also provided herein. While different genetic models may result in different scores, the results are usually correlated. Therefore the quantification of risk factors is generally not dependent on the model used.

Estimating Relative Risk Case Control Studies

A method that estimates the relative risks from the odds ratios of multiple alleles in a case-control study is also disclosed herein. In contrast to previous approaches, the method takes into consideration the allele frequencies, the prevalence of the disease, and the dependencies between the relative risks of the different alleles. The performance of the approach on simulated case-control studies was measured, and found to be extremely accurate.

Methods

In the case where a specific SNP is tested for association with a disease D, R and N denote the risk and non-risk alleles of this particular SNP. P(RR|D),P(RN|D) and P(NN|D) denote the probability of getting affected by the disease given that a person is homozygous for the risk allele, heterozygous, or homozygous for the non-risk allele respectively. f_{RR},f_{RN} and f_{NN} are used to denote the frequencies of the three genotypes in the population. Using these definitions, the relative risks are defined as

$${λ}_{RR} = \frac{P ( D \vert RR )}{P ( D \vert NN )}$$

$${λ}_{RN} = \frac{P ( D \vert RN )}{P ( D \vert NN )}$$

In a case-control study, the values P(RR|D), P(RR|˜D) can be estimated, i.e., the frequency of RR among the cases and the controls, as well as P(RN|D), P(RN|˜D), P(NN|D), and P(NN|˜D), i.e., the frequency of RN and NN among the cases and the controls. In order to estimate the relative risk, Bayes law can be used to get:

$${λ}_{RR} = \frac{P ( RR \vert D ) {f}_{NN}}{P ( NN \vert D ) {f}_{RR}}$$

$${λ}_{RN} = \frac{P ( D \vert RN ) {f}_{NN}}{P ( D \vert NN ) {f}_{RR}}$$

Thus, if the frequencies of the genotypes are known, one can use those to calculate the relative risks. The frequencies of the genotypes in the population cannot be calculated from the case-control study itself, since they depend on the prevalence of disease in the population. In particular, if the prevalence of the disease is p(D), then:

f_{RR}=P(RR|D)p(D)+P(RR|˜D)(1−p(D))

f_{RN}=P(RN|D)p(D)+P(RN|˜D)(1−p(D))

f_{NN}=P(NN|D)p(D)+P(NN|˜D)(1−p(D))

When p(D) is small enough, the frequencies of the genotypes can be approximated by the frequencies of the genotypes in the control population, but this would not be an accurate estimate when the prevalence is high. However, if a reference dataset is given (e.g., the HapMap [cite]), one can estimate the genotype frequencies based on the reference dataset.

Most current studies do not use a reference dataset to estimate the relative risk, and only the odds-ratio is reported. The odds-ratio can be written as

$$O {R}_{RR} = \frac{P ( RR \vert D ) P ( NN \vert ∼ D )}{P ( NN \vert D ) P ( RR \vert ∼ D )}$$

$$O {R}_{RN} = \frac{P ( RN \vert D ) P ( NN \vert ∼ D )}{P ( NN \vert D ) P ( RN \vert ∼ D )}$$

The odds ratios are typically advantageous since there is usually no need to have an estimate of the allele frequencies in the population; in order to calculate the odds ratios typically what is needed is the genotype frequencies in the cases and in the controls.

In some situations, the genotype data itself is not available, but the summary data, such as the odds-ratios are available. This is the case when meta-analysis is being performed based on results from previous case-control studies. In this case, how to find the relative risks from the odds ratios is demonstrated. Using the fact that the following equation holds:

p(D)=f_{RR}P(D|RR)+f_{RN}P(D|RN)+f_{NN}P(D|NN)

If this equation is divided by P(D|NN), we get

$$\frac{p ( D )}{p ( D \vert NN )} = {f}_{RR} {λ}_{RR} + {f}_{RN} {λ}_{RN} + {f}_{NN}$$

This allows the odds ratios to be written in the following way:

$$\begin{matrix}O {R}_{RR} = \frac{P ( D \vert RR ) ( 1 - P ( D \vert NN ) )}{P ( D \vert NN ) ( 1 - P ( D \vert RR ) )} \\ = {λ}_{RR} \frac{\frac{p ( D )}{p ( D \vert NN )} - p ( D )}{\frac{p ( D )}{p ( D \vert NN )} - p ( D ) {λ}_{RR}} \\ = {λ}_{RR} \frac{{f}_{RR} {λ}_{RR} + {f}_{RN} {λ}_{RN} + {f}_{NN} - p ( D )}{{f}_{RR} {λ}_{RR} + {f}_{RN} {λ}_{RN} + {f}_{NN} - p ( D ) {λ}_{RR}}\end{matrix}$$

By a similar calculation, the following system of equations results:

$$O {R}_{RR} = {λ}_{RR} \frac{{f}_{RR} {λ}_{RR} + {f}_{RN} {λ}_{RN} + {f}_{NN} - p ( D )}{{f}_{RR} {λ}_{RR} + {f}_{RN} {λ}_{RN} + {f}_{NN} - p ( D ) {λ}_{RR}} \\ O {R}_{RN} = {λ}_{RN} \frac{{f}_{RR} {λ}_{RR} + {f}_{RN} {λ}_{RN} + {f}_{NN} - p ( D )}{{f}_{RR} {λ}_{RR} + {f}_{RN} {λ}_{RN} + {f}_{NN} - p ( D ) {λ}_{RN}} \qquad Equation 1$$

If the odds-ratios, the frequencies of the genotypes in the populations, and the prevalence of the disease are known, the relative risks can be found by solving this set of equations.

Note that these are two quadratic equations, and thus they have a maximum of four solutions. However, as shown below that there is typically one possible solution to this equation.

Note that when f_{NN}=1, Equation system 1 is equivalent to the Zhang and Yu formula; however, here the allele frequency in the population is taken into account. Furthermore, our method takes into account the fact that the two relative risks depend on each other, while previous methods suggest to compute each of the relative risks independently.

Relative risks for multi-allelic loci. If multi-markers or other multi-allelic variants are considered, the calculation is complicated slightly. a₀,a₁, . . . ,aₖ is denoted by the possible k+1 alleles, where a₀ is the non-risk allele. Allele frequencies f₀,f₁,f₂, . . . ,fₖ in the population for the k+1 possible alleles are assumed. For allele i, the relative risk and odds-ratios are defined as

$${λ}_{i} = \frac{P ( D \vert {a}_{i} )}{P ( D \vert {a}_{0} )}$$

$$O {R}_{i} = \frac{P ( D \vert {a}_{i} ) ( 1 - P ( D \vert {a}_{0} ) )}{P ( D \vert {a}_{0} ) ( 1 - P ( D \vert {a}_{i} ) )} = {λ}_{i} \frac{1 - P ( D \vert {a}_{0} )}{1 - P ( D \vert {a}_{i} )}$$

The following equation holds for the prevalence of the disease:

$$p ( D ) = {\sum }_{i = 0}^{k} {f}_{i} P ( D \vert {a}_{i} )$$

Thus, by dividing both sides of the equation by p(D|a₀), we get:

$$\frac{p ( D )}{P ( D \vert {a}_{0} )} = {\sum }_{i = 0}^{k} {f}_{i} {λ}_{i} .$$

### Resulting in:

$$O {R}_{i} = {λ}_{i} \frac{{\sum }_{i = 0}^{k} {f}_{i} {λ}_{i} - p ( D )}{{\sum }_{i = 0}^{k} {f}_{i} {λ}_{i} - {λ}_{i} p ( D )} ,$$

By setting

$$C = {\sum }_{i = 0}^{k} {f}_{i} {λ}_{i} ,$$

the result is

$${λ}_{i} = C \cdot \frac{O {R}_{i}}{p ( D ) O {R}_{i} + C - p ( D )} .$$

Thus, by the definition of C, it is:

$$1 = {\sum }_{i = 0}^{k} {f}_{i} \frac{{λ}_{i}}{C} = {\sum }_{i = 0}^{k} \frac{{f}_{i} O R}{p ( D ) O {R}_{i} + C - p ( D )} .$$

This is a polynomial equation with one variable C. Once C is determined, the relative risks are determined. The polynomial is of degree k+1, and thus we expect to have at most k+1 solutions. However, since the right-hand side of the equation is a strictly decreasing as a function of C, there can typically only be one solution to this equation. A solution is then found using a binary search, since the solution is bounded between C=1 and

$$C = {\sum }_{i = 0}^{k} O {R}_{i} .$$

Robustness of the Relative Risk Estimation. The effect of each of the different parameters (prevalence, allele frequencies, and odds ratio errors) on the estimates of the relative risks was measured. In order to measure the effect of the allele frequency and prevalence estimates on the relative risk values, the relative risk was calculated from a set of values of different odds ratios, different allele frequencies (under HWE), and plotted the results of these calculations for a prevalence values ranging from 0 to 1.

Additionally, for fixed values of the prevalence, the resulting relative risks as a function of the risk-allele frequencies was plotted. Evidently, in all cases when p(D)=0, λRR=ORRR, and λRN=ORRN, and when p(D)=1, λRR=λRN=0. This can be computed directly from Equation 1. Additionally, when the risk allele frequency is high, λRR approaches a linear behavior, and λRN approaches a concave function with a bounded second derivative. When the risk-allele frequency is low, λRR and λRN approach the behavior of the function 1/p(D). This means that for high risk-allele frequency, wrong estimates of the prevalence will typically not affect the resulting relative risk by much.

Odds Ratios vs. Relative Risk. In epidemiology literature, the relative risk is often considered as an intuitive and informative measure of risk. However, the relative risk cannot be directly calculated in the context of case-control studies in general, and whole-genome association studies. The relative risk can usually be estimated through prospective studies, in which a set of healthy individuals is studied over a long period of time. In contrast, odds ratios are normally reported in case-control studies. The odds-ratio is the ratio between the odds of carrying the risk allele in the cases vs. the controls. For rare diseases, the odds ratio is a good approximation of relative risk; however, for common diseases, the odds ratio could result in a misleading estimate of risk, where the odds ratios may be quite high even when the increase in risk is minor.

Relative Lifetime Risk vs. Relative Risk. Relative risk implicitly assumes that none of the controls currently has the disease. This is relevant when the probability of having the disease is estimated. However, if interest is in the risk estimation across the span of a lifetime, or the lifetime risk of an individual to develop the condition, the fact that the some of the controls will eventually develop the disease is taken into account. The relative lifetime risk is defined as the ratio between the risk of developing the condition through the life of an individual carrying the risk allele r and the risk of developing the condition through the life of an individual carrying the non-risk allele. This is different than the standard use of relative risk in case-control studies, which is based on prevalence information.

Denoted by a₀,a₁, . . . ,aₖ is the possible k+1 alleles, where a₀ is the non-risk allele. Allele frequencies f₀,f₁,f₂, . . . ,fₖ in the population for the k+1 possible alleles are assumed. Further assumed is that studied individuals can be divided into three groups: CA, Y, and Z. CA denotes the cases, while Y and Z are controls. As opposed to individuals from Z, it is assumed that individuals from Y will eventually develop the condition. Also denoted by CO is the union of Y and Z, and by D the union of Y and CA. It is assumed that |Y|=α|CO|=αa(|Y|+|Z|), where a is the fraction of controls that will develop the condition within their lifetime. Note that α is upper bounded by the average lifetime risk. Possibly, α may be smaller than the average lifetime, depending on the age of onset of the disease, and the ages of the controls.

The relative risk and the odds ratios can now be represented as:

$${λ}_{i} = \frac{P ( CA ⋁ Y \vert {a}_{i} )}{P ( CA ⋁ Y \vert {a}_{0} )}$$

$$O {R}_{i} = \frac{P ( {a}_{i} \vert CA ) P ( {a}_{0} \vert CO )}{P ( {a}_{0} \vert CA ) P ( {a}_{i} \vert CO )}$$

The odds ratios can be written as:

$$\begin{matrix}O {R}_{i} = \frac{P ( {a}_{i} \vert CA ) P ( {a}_{0} \vert CO )}{P ( {a}_{0} \vert CA ) P ( {a}_{i} \vert CO )} \\ = \frac{P ( {a}_{i} \vert CA )}{P ( {a}_{0} \vert CA )} \cdot \frac{α P ( {a}_{0} \vert Y ) + ( 1 - α ) P ( {a}_{0} \vert Z )}{α P ( {a}_{0} \vert Y ) + ( 1 - α ) P ( {a}_{0} \vert Z )} = \\ = \frac{P ( CA \vert {a}_{i} )}{P ( CA \vert {a}_{0} )} \cdot \frac{α P ( Y \vert {a}_{0} ) + ( 1 - α ) P ( Z \vert {a}_{0} )}{α P ( Y \vert {a}_{i} ) + ( 1 - α ) P ( Z \vert {a}_{i} )} = \\ = \frac{P ( CA \vert {a}_{i} )}{P ( CA \vert {a}_{0} )} \cdot \frac{α P ( CA \vert {a}_{0} ) + ( 1 - α ) P ( Z \vert {a}_{0} )}{α P ( CA \vert {a}_{i} ) + ( 1 - α ) P ( Z \vert {a}_{i} )}\end{matrix}$$

The derivation from the first to second line is based on Bayes law, while the third line is based on the fact that CA and Y are essentially the same population, and thus P(CA|aᵢ)=P(Y|aᵢ). Now using the fact that P(Z|aᵢ)=1−P(CA|aᵢ), results in:

$$\begin{matrix}O {R}_{i} = \frac{P ( CA \vert {a}_{i} )}{P ( CA \vert {a}_{0} )} \cdot \frac{( 2 α - 1 ) P ( CA \vert {a}_{0} ) + 1 - α}{( 2 α - 1 ) P ( CA \vert {a}_{i} ) + 1 - α} \\ = {λ}_{i} \cdot \frac{( 2 α - 1 ) P ( CA \vert {a}_{0} ) + 1 - α}{( 2 α - 1 ) P ( CA \vert {a}_{i} ) + 1 - α} .\end{matrix}$$

As before,

$$p ( D ) = {\sum }_{i = 0}^{k} {f}_{i} P ( D \vert {a}_{i} ) ,$$

where p(D) is the average lifetime risk. Thus, using the equality

$$C := \frac{p ( D )}{P ( CA \vert {a}_{0} )} = {\sum }_{i = 0}^{k} {f}_{i} {λ}_{i} ,$$

and the odds ratios can be rewritten as:

$$O {R}_{i} = {λ}_{i} \cdot \frac{( 2 α - 1 ) P ( D ) + ( 1 - α ) C}{( 2 α - 1 ) P ( D ) {λ}_{i} + ( 1 - α ) C} .$$

Thus, if C is given, the relative lifetime risk can be found by assigning

$${λ}_{i} = \frac{( 1 - α ) C \cdot O {R}_{i}}{( 2 α - 1 ) P ( D ) ( 1 - O {R}_{i} ) + ( 1 - α ) C}$$

C can be found by solving the equation

$$1 = {\sum }_{i = 0}^{k} {f}_{i} \frac{{λ}_{i}}{C} = {\sum }_{i = 0}^{k} \frac{{f}_{i} ( 1 - α ) O {R}_{i}}{( 2 α - 1 ) p ( D ) ( 1 - O {R}_{i} ) + ( 1 - α ) C}$$

One can verify that by the definition of C and the odds ratios, C>(2α−1)p(D)(ORᵢ−1). Therefore, the right hand side is a decreasing function of C, and it can be found by applying a binary search.

Lifetime Risk Estimate Based on GCI. The GCI essentially provides the relative risk of an individual compared to an individual with non-risk alleles across all associated SNPs. In order to calculate the lifetime risk of an individual, the product of the lifetime risk of the individual with the average lifetime risk can be taken, and divide this product by the average lifetime risk across the population. This calculation is consistent with the definition of the average lifetime risk and of the relative risk. In order to compute the average lifetime risk, all possible genotypes are enumerated, and their relative risks that are calculated as the product of the relative risks of their variants in each of the single SNPs are summed up.

### Personalized Action Plans

The personalized action plans disclosed herein provide meaningful, actionable information to improve the health or wellness of an individual that is based on the genomic profile of the individual. The action plans provide courses of action that are beneficial to an individual in view of a particular genotype correlation, and may include administration of therapeutic treatment, monitoring for potential need of treatment or effects of treatment, or making life-style changes in diet, exercise, and other personal habits/activities, which can be personalized based on an individual's genomic profile into a personalized action plan. Alternatively, an individual may be given a particular rating that is based on their genomic profile, and in addition, optionally, include other information, such as family history, existing lifestyle habits and geography, such as, but not limited to, work conditions, work environment, personal relationships, home environment, and others. Other factors that may be incorporated include ethnicity, gender, and age. The odds ratio of various dietary and exercise prevention strategies and their association with reducing risks of diseases or conditions can also be incorporated into the rating system.

Furthermore, the personalized action plans may be modified or updated for an individual. Modified or updated personalized action plans may be automatically sent to an individual or their health care manager, for example, if an individual or their health care manager had initially requested automatic updates such as with a subscription plan. Alternatively, the updated personalized action plan may only be sent when requested by an individual or their health care manager. The personalized action plan may be modified or updated based on a number of factors. For example, an individual may have more genetic correlations analyzed and the results used to modify existing recommendations, add additional recommendations, or remove recommendations on the initial personalized action plan. In some embodiments, an individual may have changed certain lifestyle habits/environment, or have more information regarding family history, existing lifestyle habits and geography, such as, but not limited to, work conditions, work environment, personal relationships, home environment, and others, or want to include their updated age to obtain a personalized action plan that incorporates these changes. For example, an individual may have followed their initial personalized action plans, such as reducing cholesterol in their diet or pharmaceutical treatment and thus their personalized action plan recommendations may be modified or their risk or predisposition to heart disease reduced.

The personalized action plans may also have predicted future recommendations based on an individual following the recommendations on a personalized action plan or other changes an individual may make or have occur to them. For example, the individuals' increase in age would lead to an increase in risk for osteoporosis, but depending on the amount of calcium or other lifestyle habits such as those in the personalized action, the risk may be decreased.

The personalized action plan may be reported to an individual, or their health care manager, in a single report with the individual's phenotype profile and/or genomic profile. Alternatively, the personalized action plan may be reported separately. The individual can then pursue the recommended actions on their personalized action plan. The individual may choose to consult with their health care manager prior to pursuing any actions on their plan.

The personalized action plan provided can also consolidate a number of condition specific information into a consolidated set of action steps. The personalized action plan can consolidate factors including, but not limited to, the prevalence of each condition, the relative amount of pain associated with each condition, and the type of treatments for each condition. For example, if an individual has an elevated risk of myocardial infarction (for example, expressed as a higher GCI or GCI Plus score), the individual may have a personalized action plan that includes increased consumption of fruits, vegetables, and grains. However, the individual may also have a predisposition to celiac disease, thus having wheat gluten allergy. As a result, increased consumption of wheat can be contra-indicated, and is indicated in the personalized action plan.

The personalized action plan can provide pharmaceutical (which includes by definition prescription drugs, nutraceuticals and the like) recommendations, non-pharmaceutical recommendations or both. For example, the personalized action plan can include suggested pharmaceuticals as a preventative, such as cholesterol lowering drugs for an individual predisposed to myocardial infarction, and to consult with a physician. The personalized action plan can also provide non-pharmaceutical recommendations, such as following a personalized lifestyle plan, including an exercise regimen and diet plan based on an individual's genomic profile.

The personalized action plan recommendations can be of a particular rating, labeling, or categorizing system. Each recommendation may be rated or categorized by a numerical, color, and/or letter scheme or value. The recommendations may be categorized, and further rated. Numerous variations, such as different rating schemes (using letters, numbers or colors; combinations of letters, numbers, and/or colors; different types of recommendations into one or more rating schemes) may be used.

For example, an individual's genomic profile is determined and based on their genomic profile recommendations for the individual on a personalized action plan are categorized into 3 groups: “A” representing adverse or negative effects; “N” representing neutral or no significant effect, and “B” representing beneficial or positive effects. Using this system as an example, therapeutics categorized as A for the individual would include drugs that the individual has an adverse reaction to, those categorized as N would not have any significant positive or negative effect on the individual, and those categorized as B, would be beneficial to the individual's health. Using the same categorization system, a dietary plan can also be grouped into A, B, N. For example, foods which an individual is allergic to, or should particularly avoid (for example, sugars because the individual is predisposed to diabetes or cavities) would be categorized as A. Foods which have no significant effect on the individual's health may be categorized as N. Foods which are particularly beneficial to an individual may be categorized as B, for example, if an individual has high cholesterol, foods with low cholesterol would be categorized as B. Exercise regimen for the individual can also be based on the same system. For example, an individual may be predisposed to heart problems and should avoid intense workouts, and thus running may be an A activity, whereas walking or jogging at a certain pace may be categorized as a B. Standing for a period of time may be an N for one individual, but an A for another individual predisposed to varicose veins.

Furthermore, within each category of A, N, or B, there can be further levels of categorization, such as 1 through 5, from lowest to highest impact. For example, a therapeutic may be categorized as A1, which indicates a slight negative effect, such as minor nausea, whereas A2 would indicate the therapeutic would cause vomiting, while an A5 therapeutic would cause a severe adverse reaction, such as anaphylactic shock. Conversely, a B1 would have a slight positive effect on an individual, whereas B5 would have a significant positive impact on the individual. For example, if an individual is predisposed to lung cancer, or was exposed to second hand smoke while growing up, the individual not smoking may be a B5, whereas an individual not predisposed to lung cancer may have the factor as a B4.

The different categories can also be represented by different colors, for example, A can be red tones, and to represent low to high effect on an individual's health, the shades can range from a light to dark red tones, light representing low negative effects to dark red representing severe adverse effects on the individual's health. The system can also be a continuous spectrum of colors, numbers, or letters. For example, rather than have A, N, and B, and/or subcategories within, the categorization may be from A through G, wherein A represents foods, therapeutics, lifestyle habits, environments and other factors that severely negatively impact an individual's health, whereas D represents factors that have minimal effects, either positive or negative, and G would represent highly beneficial to the individual's health. Alternatively, rather than have A through G, numbers or colors may also represent the continuous spectrum of foods, therapeutics, lifestyle habits, environments and other factors that impact an individual's health.

In some embodiments, a particular therapy, pharmaceutical, or other lifestyle element in a personalized action plan can be categorized, labeled, or rated. For example, an individual may have a personalized action plan that includes an exercise regimen and a diet plan. The exercise regimen may include one or more ratings or categorization. For example, the ratings for the exercise regimen can range from A to E, such as in Table 1, wherein each letter corresponds to one or more types of exercises, including information regarding the types of activity, length of time, number of times in a given timeframe, that falls under each level, and thus, the recommended exercise regimen for the individual.

TABLE 1
Exercise Regimen: Cardiovascular Activity

| Rating | Option 1 | Option 2 | Option 3 | Option 4 |
|---|---|---|---|---|
| A | Brisk walk 2.5 mph, 3 | Swim 4 laps, 3 | Cycle 5 mph, 3 times a | Brisk walk 2.5 mph, 2 times a |
| | times a week, for 20 | times a week | week, for 20 minutes | week, for 20 minutes |
| | minutes | | | Cycle 5 mph, once a week, for 20 |
| | | | | minutes |
| B | Jog 3.5 mph, 3 times a | Swim 6 laps, 3 | Cycle 8 mph, 3 times a | Jog 3.5 mph, 2 times a week, for |
| | week, for 20 minutes | times a week | week, for 20 minutes | 20 minutes |
| | | | | Cycle 8 mph once a week, for 20 |
| | | | | minutes |
| C | Run 4 mph, 3 times a | Swim 8 laps, 3 | Cycle 10 mph, 3 times | Run 4 mph 2.5 mph, 2 times a |
| | week, for 20 minutes | times a week | a week, for 20 minutes | week for 20 minutes |
| | | | | Cycle 10 mph, once a week, for 20 |
| | | | | minutes |
| D | Run 5 mph, 3 times a | Swim 10 laps, 3 | Cycle 15 mph, 3 times | Run 5 mph, 2 times a week for 25 |
| | week, for 25 minutes | times a week | a week, for 30 minutes | minutes |
| | | | | Cycle 15 mph, once a week, for 20 |
| | | | | minutes |
| E | Run 6 mph 3 times a | Swim 12 laps, 3 | Cycle 15 mph, 3 times | Run 5 mph, 2 times a week for 30 |
| | week, for 30 minutes | times a week | a week, for 40 minutes | minutes |
| | | | | Cycle 15 mph, once a week, for 40 |
| | | | | minutes |

In one embodiment, based on the genomic profile of the individual, the personalized action plan may having an A rating for an individual, and therefore the individual's recommended exercise regimen would be to select from the choices in Row A in Table 1 for their cardiovascular workout. Similarly, an analogous system for weight training can be part of the individual's exercise regimen, and weight training options for an A rating would be recommended for the individual. In some embodiments, factors such as, but not limited to, an individual's existing diet, exercise, and other personal habits/activities, optionally, other information, such as family history, existing lifestyle habits and geography, such as, but not limited to, work conditions, work environment, personal relationships, home environment, ethnicity, gender, age, and other factors may be incorporated with an individual's genomic profile determine the individual's exercise regiment rating. Furthermore, as an individual's lifestyle habits changes, or more factors become known and are incorporated, the individual's rating can change, for example, if an individual follows the recommended activities on the personalized action plan, starting at an A rating, the individual may request an updated personalized action plan that evaluates and determines the individual is now at a B rating. Alternatively, an individual's personalized action plan may offer a timeline for when the individuals should consider moving from an A rating to a B rating to maximize their health.

The personalized action plan may also have a rating system for a dietary plan. For example, the ratings for the dietary plan can be a system that ranges from 1 to 5, wherein each number corresponds to particular grouping of fats, fibers, proteins, sugars, and other nutrients the individual is suggested to have in their diet, particular portion sizes, number of calories, and/or grouping with other foods that an individual should have as their diet. Based on the genomic profile of the individual, the personalized action plan may give a 2 rating for an individual, and therefore the individual's recommended dietary plan would be a selection of dietary choices under a 2 rating.

In another embodiment, individual foods may be categorized. For example, an individual given a 2 rating should select specific foods that are also categorized as 2. For example, specific vegetables, meats, fruits, diary, and others may be categorized as a 2, while others not. For example, asparagus may be a vegetable that is a 2, whereas beets are a 3, and therefore the individual should include more asparagus rather than beets in their diet.

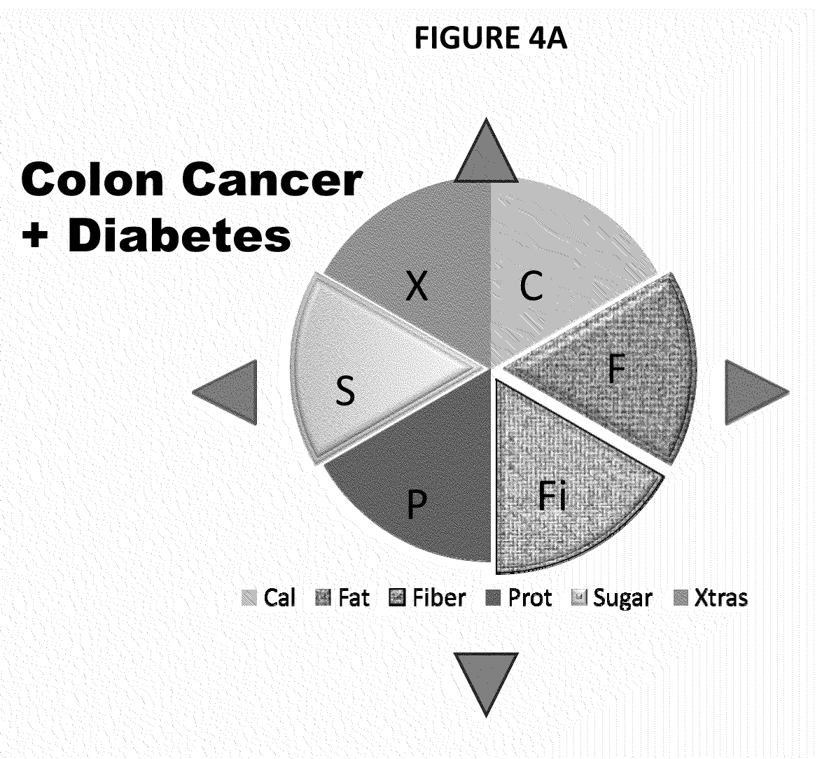

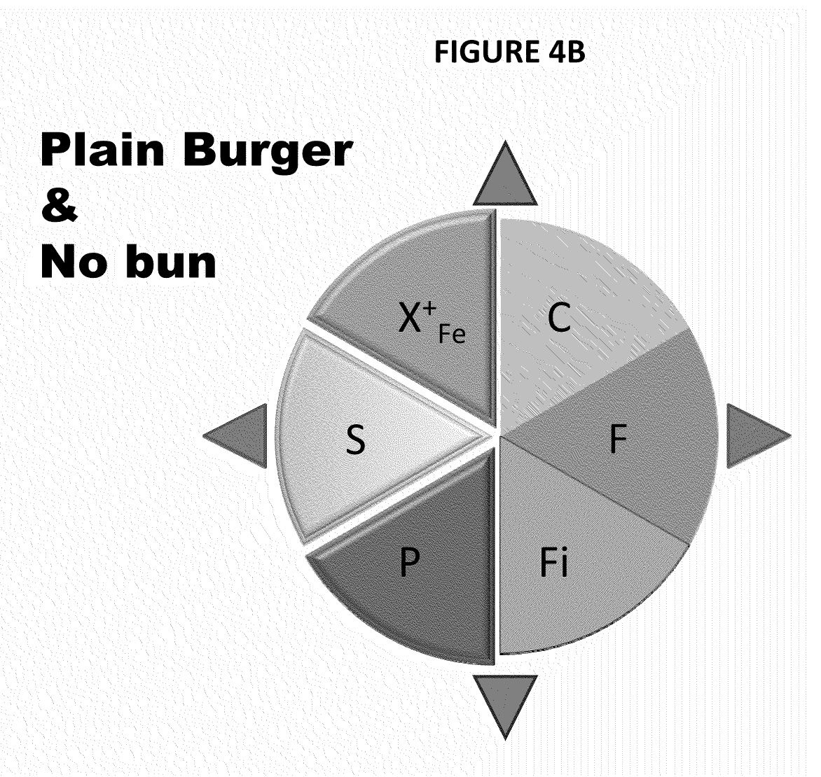

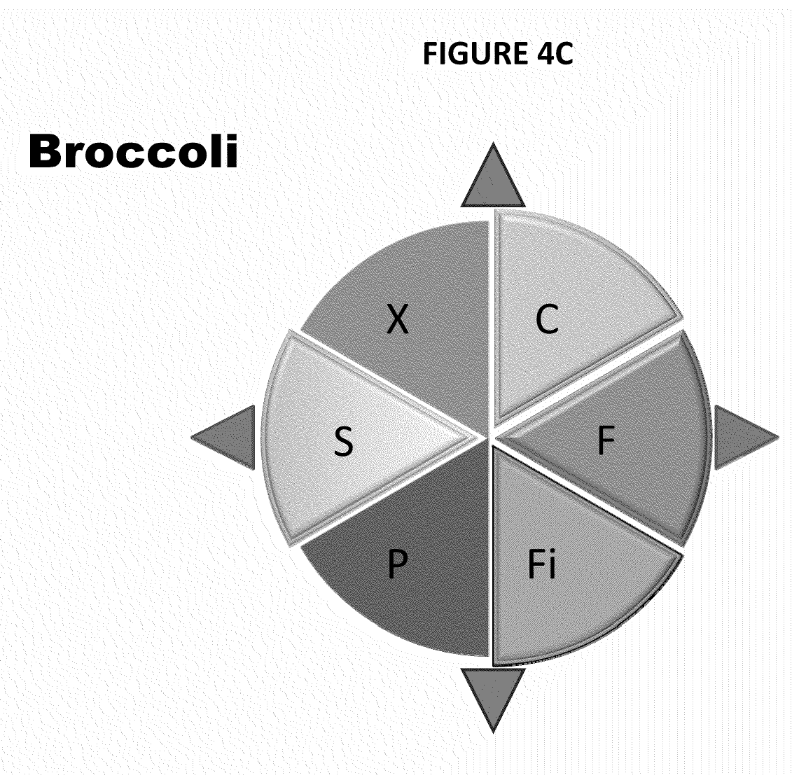

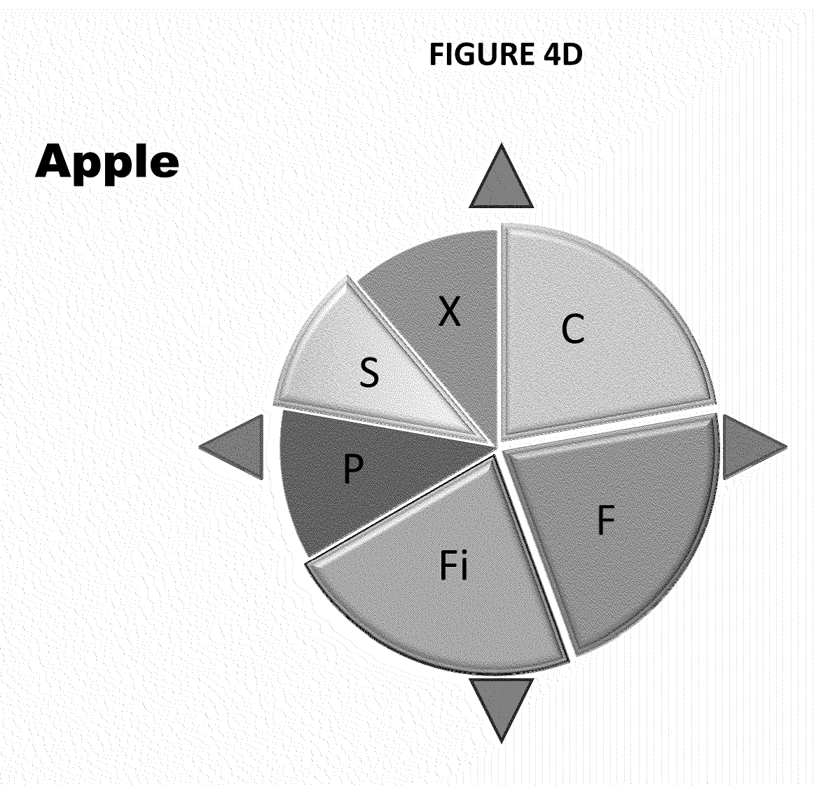

In another embodiment, an individual is given a suggested rating for what type of diet to follow that is breakdown of the types of nutrients of the type of food the individual should have in their diet, based on their genomic profile. The rating may be in the form of a visual representation that includes shapes, colors, numbers, and/or letters. The rating may be in the form of a visual representation that includes shapes, colors, numbers, and/or letters. For example, an individual is found to be predisposed to colon cancer and diabetes, and is given a symbol that represent the proportion of different nutrients in the recommended types of food the individual should have in their diet, as shown in FIG. 4A (see also Example 3). Different types of foods, such as, but not limited to, specific fruits, vegetables, carbohydrates, meats, diary products, and the like are represented by the same scheme, such as shown in FIGS. 4B-4D. Foods with rated with a symbol that most closely resembles that given the individual, such as depicted FIG. 4A, would be recommended foods for the individual.

In some embodiments, factors such as, but not limited to, an individual's existing diet, exercise, and other personal habits/activities, optionally, other information, such as family history, existing lifestyle habits and geography, such as, but not limited to, work conditions, work environment, personal relationships, home environment, ethnicity, gender, age, and other factors may be incorporated with an individual's genomic profile to create a personalized action plan, and thus affect the rating given for the individual's dietary plan. Furthermore, as an individual's lifestyle habits changes, or more factors become known and are incorporated, the individual's rating can change. For example, if an individual follows the recommended activities on the personalized action plan, starting at a 1 rating for dietary plans, which is an extremely low cholesterol diet, the individual may request an updated personalized action plan that incorporates the changes in lifestyle habits the individual has had such that the individual has an improved cholesterol level, the updated personalized action plan may show that the individual may be better suited to now follow dietary plans under rating 2, or can choose from dietary plans in ratings 1 and 2. Alternatively, an individual's initial personalized action plan can offer a timeline for when the individuals should consider moving from a 1 rating to a 2 rating, or vary their dietary plans based on a schedule, between different dietary plans under different ratings, to maximize their health.

The ratings in a personalized action plan may be for a combination of different rating systems. For example, an exercise regimen system with ratings A through E and dietary plan system with ratings 1 through 5 can be used to give an individual an A1 rating in their personalized action plan. Therefore, the individual is recommended to follow the exercise regimen of the A rating and the dietary plan of the 1 rating. Alternatively, a single rating system can be used for the exercise and diet regimen. For example, an individual may be given a particular rating such as a C rating in a personalized action plan such that the recommended exercise and dietary regimen for the individual is both under the C categorization. In other embodiments, other types of recommendations, such as other lifestyle activities and habits, are also included. For example, other than exercise and dietary regimens, other recommendations, such as therapeutics, type of work environment, type of social activities, can also be encompassed under a singe rating system. Alternatively, different rating systems can be used for other recommendations. For example, letters may be used for recommended exercise regimen, numbers for dietary regimen, and colors for pharmaceutical recommendations.

In some embodiments a binary rating systems is used, such that types of recommendations are grouped into pairs. The system can be similar to the Myers Briggs Type Indicator (MBTI) system. In the MBTI system, there are four pairs of preferences or dichotomies, and an individual is placed into one of each pair. An individual's preference is 1) extraversion or introversion, 2) sensing or intuition, 3) thinking or feeling, and 4) judging or perceiving. A variation in the system can be used in determining recommendations for an individual to improve their health and well-being that is based on an individual's genomic profile.

For example, an individual may be either an A or a B for diets, wherein A represents a certain type of mix of nutrients and B is a different mix. Alternatively, specific types of foods may be grouped into A or B. The individual may have another binary categorization for exercise regimen, such as H or L, where H represents that an individual should participate in high-impact exercise, and L represent low-impact activities. As such, an individual may be categorized as an AH. Another binary categorization can be for social contact. For example, an individual can be genetically predisposed to being social (S) or unsocial (U), and as such, recommendations may include the type of activities or groups of people the individual should avoid or seek to reduce stress and increase their health and well-being.

The personalized action plans can also be updated to include factors based on information as they become known, including scientific information, or information from the individual, such as “field-deployed” or direct mechanisms, for example, metabolite levels, glucose levels, ion levels (for example, calcium, sodium, potassium, iron), vitamins, blood cell counts, body mass index (BMI), protein levels, transcript levels, heart rate, etc., can be determined by methods readily available and can be factored into the personalized action plan when they are known, as they become known, such as by real time monitoring. The personalized action plan can be modified, for example, based on an individual following the plan, which may also affect the predisposition an individual may have for one or more conditions. For example, the GCI score of the individual may be updated.

### Communities and Motivations

The present disclosure provides phenotype profiles and personalized action plans that are based on an individual's genomic profile, such that individuals are well informed about their health and well-being, and the customized options individuals have to improve their health. Also provided herein are communities, such as on-line communities, that can offer support and motivation for an individual to pursue their personalized action plan. Motivation for individuals to improve their health, for example, by following their personalized action plan, can also include financial incentives.

An individual may participate in a community, such as an on-line community, where the individual or their health care manager has access to the individual's genomic profile, phenotype profile, and/or personalized action plan. The individual may choose to have genomic profile, phenotype profile, and/or personalized action plan available for all of the community, a subset of the community, or none of the community to view, through a personal on-line portal. Friends, family, or co-workers may be part of the on-line community. For example, on-line communities such as https://changefire.com are known in the arts, for motivating individuals to achieve their goals. In the present disclosure, an individual participates or is a member of an on-line community that supports and motivates an individual to improve their health and well-being, using as a baseline their phenotype profile, such as GCI scores or by achieving goals on their personalized action plan. The on-line community may be limited to an individual's friends, family, or co-workers, or a combination of friends, family, and co-workers. The individual may also include other members of the on-line community they had not known previously. The on-line community may also be an employer sponsored community. The individual may form groups with others with similar phenotype profiles, action plans, and motivate each other to achieve their goals. Individuals may set up competitions with others in the on-line community, to improve their GCI scores and/or achieve goals on their personalized action plan.

For example, an individual's report, such as their GCI scores and personalized action plan, may be viewable by an individual's family and friends in the on-line community. An individual may have the choice or option of selecting who may view and/or access their report. The on-line version may comprise a checklist or milestone measure containing items on the personalized action plan, where the individual may mark off accomplishments or the progress of their personalized action plan. The GCI scores may be updated as the genetic information changes and reflected on the report on-line. The individual may also input factors that may have changed, such as lifestyle changes, exercise regimen changes, dietary changes, pharmaceutical treatment(s) and others, which may also alter the report for the individual. Family and friends may view the progress of the individual, as well as changes in the individual's life, and how they may reflect or alter the individual's risks or predisposition. The on-line portal may allow the individual view initial and subsequent reports. The individual may also receive feedback and comments from their friends and family. Family and friends may leave supporting and motivating comments.

The on-line community can also provide incentives for an individual to improve their health, by progressing through their personalized action plan, and/or, decreasing their risk or predisposition to diseases. Incentives can also be provided to individuals not in an on-line community. For example, an employer sponsored online community may offer a health plan that the employer subsidizes more of, provide extra vacation days, or contribute to the health savings account of the individual, when the individual reaches certain goals, such as by progressing through their personalized action plan, thereby decreasing their risks and/or predisposition to a disease. Alternatively, the community does not have to be online, and the individual submits evidence of their progression to a designated person that processes the health plans for the employer through their personalized action plan and/or decreased disposition for disease.

Other incentives may also be used to motivate an individual to improve their health by decreasing their predisposition for disease, and/or following their personalized action plan. Individuals may receive points to redeem for rewards when they reach certain goals, such as decreasing their risk for disease by a certain percentage or numerical value, or moving from one category to another (i.e. higher risk to lower risk), or by achieving certain goals in the personalized action plan. For example, the individual may achieve a risk decrease of a certain numerical value, to achieve the greatest decrease in risk to a disease within a certain timeframe, to accomplish a goal on the personalized action plan, or to accomplish the most goals on a personalized action plan.

Friends, family, and/or employers may offer points and/or rewards, perhaps by purchasing them, and offering them as a reward to the individual that decreases their risks or predisposition for disease and/or achieves goals on their personalized action plan. Individuals may also receive points/awards for reaching a goal before another person, such as another co-worker, or group of friends, family, or members of an on-line community with the same goal. For example, the first to achieve a risk decrease of a certain numerical value, to achieve the greatest decrease in risk to a disease within a certain timeframe, to accomplish a goal on the personalized action plan, or to accomplish the most goals on a personalized action plan. The individual may receive cash, or points to redeem for cash, as rewards. Other rewards may include pharmaceutical products, health products, health club memberships, spa treatments, medical procedures, devices to monitor health, genetic tests, trips, and others, such as subscriptions to services described herein, or discounts, subsidies or reimbursements for the aforementioned items.

The incentives may be sponsored by friends, family, and employers. Pharmaceutical companies, health clubs, medical device companies, spas, and others may also sponsor incentives. The sponsorship may be in exchange for advertising, or recruiting, for example, pharmaceutical companies may be interested in obtaining the genome profile of individuals for data, or clinical trials. Furthermore, the incentives may be used to encourage individuals to participate in communities that motivate individuals to improve their health, such as the on-line communities described herein.

### Accessing Profiles and Personalized Action Plans

Reports containing the genomic profile, phenotype profile and other information related to the phenotype and genomic profiles, such as personalized action plans, may be provided to the individual. Health care managers and providers, such as caregivers, physicians, and genetic counselors may also have access to the reports. The reports may be printed, saved on the computer, or viewed on-line. Alternatively, the profiles and action plans may be provided in paper form. They may be in paper, or computer readable format, such as online at a certain time, with subsequent updates provided by paper, computer readable format, or online. The profiles and action plans can be encoded on a computer readable medium.

The genomic profile, phenotype profile, as well as personalized action plans can be accessible by an on-line portal, a source of information which can be readily accessed by an individual through use of a computer and internet website, telephone, or other means that allow similar access to information. The on-line portal may optionally be a secure on-line portal or website. It may provide links to other secure and non-secure websites, for example links to a secure website with the individual's phenotype profile, or to non-secure websites such as a message board for individuals sharing a specific phenotype.

Reports may be of an individual's GCI score, or GCI Plus score (as described herein, to report a GCI score will also encompass methods of reporting a GCI Plus score or both). For example, the score, for one or more conditions, can be visualized using a display. A screen (such as a computer monitor or television screen) can be used to visualize the display, such as a personal portal with relevant information. In another embodiment, the display is a static display such as a printed page. The display may include, but is not limited to, one or more of the following: bins (such as 1-5, 6-10, 11-15, 16-20, 21-25, 26-30, 31-35, 36-40, 41-45, 46-50,51-55, 56-60, 61-65, 66-70, 71-75, 76-80, 81-85, 86-90, 91-95, 96-100), a color or grayscale gradient, a thermometer, a gauge, a pie chart, a histogram or a bar graph. In another embodiment, a thermometer is used to display the GCI score and disease/condition prevalence. The thermometer can display a level that changes with the reported GCI score, for example, the thermometer may display a colorimetric change as the GCI score increases (such as changing from blue, for a lower GCI score, progressively to red, for a higher GCI score). In a related embodiment a thermometer displays both a level that changes with the reported GCI score and a colorimetric change as the risk rank increases

An individual's GCI score can also be delivered to an individual by using auditory feedback. For example, the auditory feedback can be a verbalized instruction that the risk rank is high or low. The auditory feedback can also be a recitation of a specific GCI score such as a number, a percentile, a range, a quartile or a comparison with the mean or median GCI score for a population. In one embodiment, a live human delivers the auditory feedback in person or over a telecommunications device, such as a phone (landline, cellular phone or satellite phone) or via a personal portal. The auditory feedback can also be delivered by an automated system, such as a computer. The auditory feedback can be delivered as part of an interactive voice response (IVR) system, which is a technology that allows a computer to detect voice and touch tones using a normal phone call. An individual may interact with a central server via an IVR system. The IVR system may respond with pre-recorded or dynamically generated audio to interact with individuals and provide them with auditory feedback of their risk rank. An individual may call a number that is answered by an IVR system. After optionally entering an identification code, a security code or undergoing voice-recognition protocols the IVR system may asks the individual to select options from a menu, such as a touch tone or voice menu. One of these options may provide an individual with his or her risk rank.

An individual's GCI score may be visualized using a display and delivered using auditory feedback, such as over a personal portal. This combination may include a visual display of the GCI score and auditory feedback, which discusses the relevance of the GCI score to the individual's overall health and possible preventive measures, such as their personalized action plan.

Different report options may be accessible to the individual. For example, an online access point, such as an online portal may allow an individual to display a single phenotype, or more than one phenotype, based on their genomic profile. The subscriber may also have different viewing options, for example, such as a “Quick View” option, to give a brief synopsis of a single or multiple conditions. A “Comprehensive View” option may also be selected, where more detail for each category is provided. For example, there may be more detailed statistics about the likelihood of the individual developing the phenotype, more information about the typical symptoms or phenotypes, such as sample symptoms for a medical condition, or the range of a physical non-medical condition such as height, or more information about the gene and genetic variant, such as the population incidence, for example in the world, or in different countries, or in different age ranges or genders. For example, a summary of estimated lifetime risks for a number of conditions may be in a “Quick View” option, while more information for a specific condition, such as prostate cancer or Crohn's disease may be other viewing options. Different combinations and variations may exist for different viewing options.

The phenotype selected by an individual can be a medical condition and different treatments and symptoms in the report may link to other web pages that contain further information about the treatment. For example, by clicking on a drug, it will lead to website that contains information about dosages, costs, side effects, and effectiveness. It may also compare the drug to other treatments. The website may also contain a link leading to the drug manufacturer's website. Another link may provide an option for the subscriber to have a pharmacogenomic profile generated, which would include information such as their likely response to the drug based on their genomic profile. Links to alternatives to the drug may also be provided, such as preventative action such as fitness and weight loss, and links to diet supplements, diet plans, and to nearby health clubs, health clinics, health and wellness providers, day spas and the like may also be provided. Educational and informational videos, summaries of available treatments, possible remedies, and general recommendations may also be provided.

The on-line report may also provide links to schedule in-person physician or genetic counseling appointments or to access an on-line genetic counselor or physician, providing the opportunity for a subscriber to ask for more information regarding their phenotype profile. Links to on-line genetic counseling and physician questions may also be provided on the on-line report.

In another embodiment, the report may be of a “fun” phenotype, such as the similarity of an individual's genomic profile to that of a famous individual, such as Albert Einstein. The report may display a percentage similarity between the individual's genomic profile to that of Einstein's, and may further display a predicted IQ of Einstein and that of the individual's. Further information may include how the genomic profile of the general population and their IQ compares to that of the individual's and Einstein's.

In another embodiment, the report may display all phenotypes that have been correlated to the individual's genomic profile. In other embodiments, the report may display only the phenotypes that are positively correlated with an individual's genomic profile. In other formats, the individual may choose to display certain subgroups of phenotypes, such as only medical phenotypes, or only actionable medical phenotypes. For example, actionable phenotypes and their correlated genotypes, may include Crohn's disease (correlated with IL23R and CARD 15), Type 1 diabetes (correlated with HLA-DR/DQ), lupus (correlated HLA-DRB1), psoriasis (HLA-C), multiple sclerosis (HLA-DQA1), Graves disease (HLA-DRB1), rheumatoid arthritis (HLA-DRB1), Type 2 diabetes (TCF7L2), breast cancer (BRCA2), colon cancer (APC), episodic memory (KIBRA), and osteoporosis (COL1A1). The individual may also choose to display subcategories of phenotypes in their report, such as only inflammatory diseases for medical conditions, or only physical traits for non-medical conditions. In some embodiments, the individual may choose to show all conditions an estimated risk was calculated for the individual by highlighting those conditions, highlighting only conditions with an elevated risk, or only conditions with a reduced risk.

Information submitted by and conveyed to an individual may be secure and confidential, and access to such information may be controlled by the individual. Information derived from the complex genomic profile may be supplied to the individual as regulatory agency approved, understandable, medically relevant and/or high impact data. Information may also be of general interest, and not medically relevant. Information can be securely conveyed to the individual by several means including, but not restricted to, a portal interface and/or mailing. More preferably, information is securely (if so elected by the individual) provided to the individual by a portal interface, to which the individual has secure and confidential access. Such an interface is preferably provided by on-line, internet website access, or in the alternative, telephone or other means that allow private, secure, and readily available access. The genomic profiles, phenotype profiles, and reports are provided to an individual or their health care manager by transmission of the data over a network.

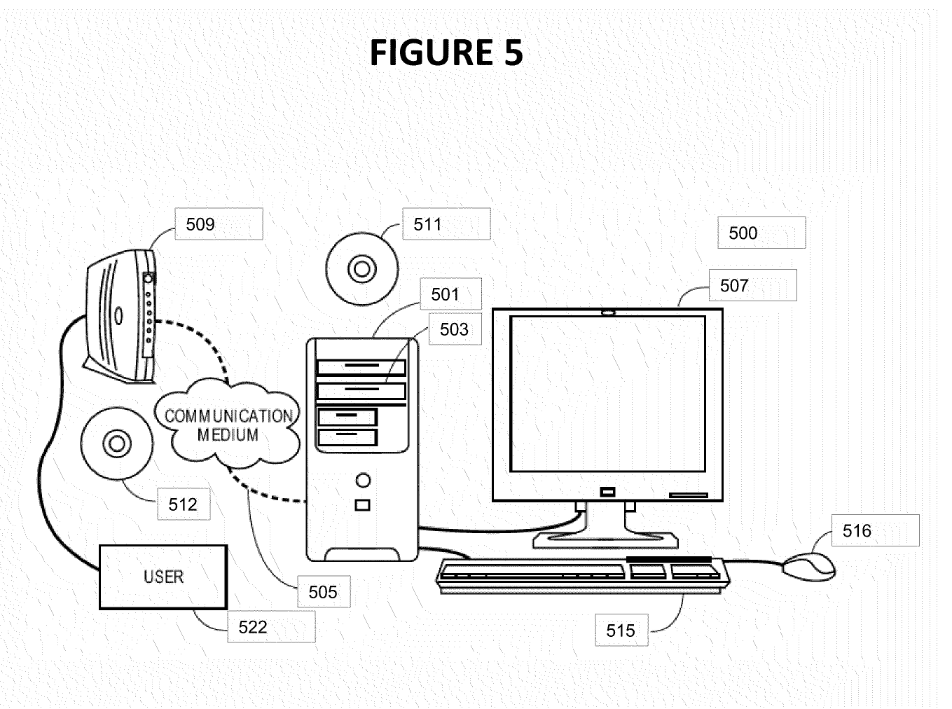

Accordingly, a representative example logic device through which a report may be generated can comprise a computer system (or digital device), such as shown in FIG. 5 (500). The computer system can receive and store genomic profiles, analyze genotype correlations, generate rules based on the analysis of genotype correlations, apply the rules to the genomic profiles, and produce a phenotype profile, a personalized action plan, and report. For example, personalized action plan can be obtained and outputted from the computer system. The computer system 500 may be understood as a logical apparatus that can read instructions from media 511 and/or a network port 505, which can optionally be connected to server 509 having fixed media 512. The system, such as shown in FIG. 5 can include a CPU 501, disk drives 503, optional input devices such as keyboard 515 and/or mouse 516 and optional monitor 507. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 522. The receiving party 522 can be but is not limited to an individual, a health care provider or a health care manager. In one embodiment, a computer-readable medium includes a medium suitable for transmission of a result of an analysis of a biological sample or a genotype correlation. The medium can include a result regarding a phenotype profile of an individual and/or an action plan for the individual, wherein such a result is derived using the methods described herein.

A personal portal can serve as the primary interface with an individual for receiving and evaluating genomic data. A portal can enable individuals to track the progress of their sample from collection through testing and results. Through portal access, individuals are introduced to relative risks for common genetic disorders based on their genomic profile. The individual may choose which rules to apply to their genomic profile through the portal.

In one embodiment, one or more web pages will have a list of phenotypes and next to each phenotype a box in which a subscriber may select to include in their phenotype profile. The phenotypes may be linked to information on the phenotype, to help the subscriber make an informed choice about the phenotype they want included in their phenotype profile. The webpage may also have phenotypes organized by disease groups, for example as actionable diseases or not. For example, an individual may choose actionable phenotypes only, such as HLA-DQA1 and celiac disease. The subscriber may also choose to display pre or post symptomatic treatments for the phenotypes. For example, the individual may choose actionable phenotypes with pre-symptomatic treatments (outside of increased screening), for celiac disease, a pre-symptomatic treatment of gluten free diet. Another example may be for Alzheimer's, the pre-symptomatic treatment of statins, exercise, vitamins, and mental activity. Thrombosis is another example, with a pre-symptomatic treatment of avoiding oral contraceptives and avoiding sitting still for long periods of time. An example of a phenotype with an approved post symptomatic treatment is wet AMD, correlated with CFH, wherein individuals may obtain laser treatment for their condition.

The phenotypes may also be organized by type or class of disease or conditions, for example neurological, cardiovascular, endocrine, immunological, and so forth. Phenotypes may also be grouped as medical and non-medical phenotypes. Other groupings of phenotypes on the webpage may be by physical traits, physiological traits, mental traits, or emotional traits. The webpage may further provide a section in which a group of phenotypes are chosen by selection of one box. For example, a selection for all phenotypes, only medically relevant phenotypes, only non-medically relevant phenotypes, only actionable phenotypes, only non-actionable phenotypes, different disease group, or “fun” phenotypes. “Fun” phenotypes may include comparisons to celebrities or other famous individuals, or to other animals or even other organisms. A list of genomic profiles available for comparison may also be provided on the webpage for selection by the individual to compare to the individual's genomic profile.

The on-line portal may also provide a search engine, to help the individual navigate the portal, search for a specific phenotype, or search for specific terms or information revealed by their phenotype profile or report. Links to access partner services and product offerings may also be provided by the portal. Additional links to support groups, message boards, and chat rooms for individuals with a common or similar phenotype may also be provided. The on-line portal may also provide links to other sites with more information on the phenotypes in an individual's phenotype profile. The on-line portal may also provide a service to allow individuals to share their phenotype profile and reports with friends, families, co-workers, or health care managers, and may choose which phenotypes to show in the phenotype profile they want shared with their friends, families, co-workers, or health care managers.

The phenotype profiles and reports provide a personalized genotype correlation to an individual. The genotype correlations used to generate a personalized action plan that provides individuals with increased knowledge and opportunities to determine their personal health care and lifestyle choices. If a strong correlation is found between a genetic variant and a disease for which treatment is available, detection of the genetic variant may assist in deciding to begin treatment of the disease and/or monitoring of the individual. In the case where a statistically significant correlation exists but is not regarded as a strong correlation, an individual can review the information with a personal physician and decide an appropriate, beneficial course of action. Potential courses of action that could be beneficial to an individual in view of a particular genotype correlation include administration of therapeutic treatment, monitoring for potential need of treatment or effects of treatment, or making life-style changes in diet, exercise, and other personal habits/activities, which can be personalized based on an individual's genomic profile into a personalized action plan. Other personal information, such as existing habits and activities can also be incorporated into a personalized action plan. For example, an actionable phenotype such as celiac disease may have a pre-symptomatic treatment of a gluten-free diet, and which may be provided in a personalized action plan. Likewise, genotype correlation information could be applied through pharmacogenomics to predict the likely response an individual would have to treatment with a particular drug or regimen of drugs, such as the likely efficacy or safety of a particular drug treatment.

Genotype correlation information can also be used in cooperation with genetic counseling to advise couples considering reproduction, and potential genetic concerns to the mother, father and/or child. Genetic counselors may provide information and support to individuals with phenotype profiles that display an increased risk for specific conditions or diseases. They may interpret information about the disorder, analyze inheritance patterns and risks of recurrence, and review available options with the subscriber. Genetic counselors may also provide supportive counseling refer subscribers to community or state support services. Genetic counseling may be included with specific subscription plans. Genetic counseling options can also include those that are scheduled within 24 hours of request and available during non-traditional hours, such as evenings, Saturdays, Sundays, and/or holidays.

An individual's portal can also facilitate delivery of additional information beyond an initial screening. Individuals can be informed about new scientific discoveries that relate to their personal genetic profile, such as information on new treatments or prevention strategies for their current or potential conditions. The new discoveries may also be delivered to their healthcare managers. The new discoveries can be incorporated into updated or revised personal action plans. The individuals or their healthcare providers can be informed of new genotype correlations and new research about the phenotypes in the individual's phenotype profiles by e-mail. For example, e-mails of “fun” phenotypes can be sent to individuals, for example, an e-mail may inform them that their genomic profile is 77% identical to that of Abraham Lincoln and that further information is available via an on-line portal.

Computer code for notifying subscribers of new or revised correlations new or revised rules, and new or revised reports, for example with new prevention and wellness information, information about new therapies in development, or new treatments available, is also provided herein. A system of computer code for generating new rules, modifying rules, combining rules, periodically updating the rule set with new rules, maintaining a database of genomic profile securely, applying the rules to the genomic profiles to determine phenotype profiles, generating personalized action plans and reports is also provided by the present disclosure, including computer code for granting different levels of access and options for individuals with different subscriptions.

### Subscriptions

The genomic profiles, phenotype profiles, and reports, including personalized action plans may be generated, such as by a computer, for individuals that are human or non-human. For example, individuals may include other mammals, such as bovines, equines, ovines, canines, or felines. An individual may be a person's pet, and the owner of the pet may want a personal action plan to increase the health and longevity of their pet. Individuals, or their health care managers, may be subscribers. As described herein, subscribers are human individuals who subscribe to a service by purchase or payment for one or more services. Services may include, but are not limited to, one or more of the following: having their or another individual's, such as the subscriber's child or pet, genomic profile determined, obtaining a phenotype profile, having the phenotype profile updated, and obtaining reports based on their genomic and phenotype profile, including a personalized action plan.

Subscribers may choose to provide the genomic and phenotype profiles or reports to their health care managers, such as a physician or genetic counselor. The genomic and phenotype profiles may be directly accessed by the healthcare manager, by the subscriber printing out a copy to be given to the healthcare manager, or have it directly sent to the healthcare manager through the on-line portal, such as through a link on the on-line report.

A genomic profile may be generated for subscribers and non-subscribers and stored digitally, such as on a computer readable medium, but access to the phenotype profile and reports, such as outputted through a computer, may be limited to subscribers. For example, access to at least one GCI score generated and outputted by a computer is provided to a subscriber, but not to non-subscribers. In another variation, both subscribers and non-subscribers may access their genotype and phenotype profiles with a computer, but have limited access, or have a limited report generated for non-subscribers, whereas subscribers have full access and may have a full report generated. In another embodiment, both subscribers and non-subscribers may have full access initially, or full initial reports, but only subscribers may access updated reports based on their stored genomic profile. For example, access is provided to non-subscribers, where they may have limited access to at least one of their GCI scores, or they may have an initial report on at least one of their GCI scores generated, but updated reports are generated only with purchase of a subscription. Health care managers and providers, such as caregivers, physicians, and genetic counselors may also have access to at least one of an individual's GCI scores.

Other subscription models may include one that provides a phenotype profile where the subscriber may choose to apply all existing rules to their genomic profile, or a subset of the existing rules, to their genomic profile. For example, they may choose to apply only the rules for disease phenotypes that are actionable. The subscription may be of a class, such that there are different levels within a single subscription class. For example, different levels may be dependent on the number of phenotypes a subscriber wants correlated to their genomic profile, or the number of people that may access their phenotype profile.

Another level of subscription may be to incorporate factors specific to an individual, such as already known phenotypes such as age, gender, or medical history, to their phenotype profile. Still another level of the basic subscription may allow an individual to generate at least one GCI score for a disease or condition. A variation of this level may further allow an individual to specify for an automatic update of at least one GCI score for a disease or condition to be generated if their is any change in at least one GCI score due to changes in the analysis used to generate at least one GCI score. In some embodiments the individual may be notified of the automatic update by email, voice message, text message, mail delivery, or fax.

Subscribers may also generate reports that have their phenotype profile as well as information about the phenotypes, such as genetic and medical information about the phenotype. Different amount of information that an individual may access can depend on the level of subscription they have. For example, different viewing options an individual may have could depend on their level of subscription, such as a quick view for non-subscribers or a more basic subscription, but a comprehensive view is accessible to those with a full subscription.

For example, different levels of subscriptions may have different variations or combinations of accessibility to information including, but not limited to, the prevalence of the phenotype in the population, the genetic variant that was used for the correlation, the molecular mechanism that causes the phenotype, therapies for the phenotype, treatment options for the phenotype, and preventative actions, may be included in the report. In other embodiments, the reports may also include information such as the similarity between an individual's genotype and that of other individuals, such as celebrities or other famous people. The information on similarity may be, but not limited to, percentage homology, number of identical variants, and phenotypes that may be similar. These reports may further contain at least one GCI score.

Other options based on subscription level may include links to other sites with further information on the phenotypes, links to on-line support groups and message boards of people with the same phenotype or one or more similar phenotypes, links to an on-line genetic counselor or physician, or links to schedule telephonic or in-person appointments with a genetic counselor or physician, if the report is accessed on-line. If the report is in paper form, the information may be the website location of the aforementioned links, or the telephone number and address of the genetic counselor or physician. The subscriber may also choose which phenotypes to include in their phenotype profile and what information to include in their report. The phenotype profile and reports may also be accessible by an individual's health care manager or provider, such as a caregiver, physician, psychiatrist, psychologist, therapist, or genetic counselor. The subscriber may be able to choose whether the phenotype profile and reports, or portions thereof, are accessible by such individual's health care manager or provider.

Another level of subscription may be to maintain the genomic profile of an individual digitally after generation of an initial phenotype profile and report, and provides subscribers the opportunity to generate phenotype profiles and reports with updated correlations from the latest research. Subscribers may have the opportunity to generate risk profile and reports with updated correlations from the latest research. As research reveals new correlations between genotypes and phenotypes, disease or conditions, new rules will be developed based on these new correlations and can be applied to the genomic profile that is already stored and being maintained. The new rules may correlate genotypes not previously correlated with any phenotype, correlate genotypes with new phenotypes, modify existing correlations, or provide the basis for adjustment of a GCI score based on a newly discovered association between a genotype and disease or condition. Subscribers may be informed of new correlations via e-mail or other electronic means, and if the phenotype is of interest, they may choose to update their phenotype profile with the new correlation. Subscribers may choose a subscription where they pay for each update, for a number of updates or an unlimited number of updates for a designated time period (e.g. three months, six months, or one year). Another subscription level may be where a subscriber has their phenotype profile or risk profile automatically updated, instead of where the individual chooses when to update their phenotype profile or risk profile, whenever a new rule is generated based on a new correlation.

Subscribers may also refer non-subscribers to the service that generates rules on correlations between phenotypes and genotypes, determines the genomic profile of an individual, applies the rules to the genomic profile, and generates a phenotype profile of the individual. Referral by a subscriber may give the subscriber a reduced price on subscription to the service, or upgrades to their existing subscriptions. Referred individuals may have free access for a limited time or have a discounted subscription price.

The following examples illustrate and explain the embodiments described herein. The scope of the disclosure is not limited by these examples.

### EXAMPLES

### Example 1

### A Comparison Between the GCI Scores

The CCI score is calculated based on multiple models across the HapMap CEU population, for 10 SNPs associated with T2D. The relevant SNPs were rs7754840, rs4506565, rs7756992, rs10811661, rs12804210, rs8050136, rs1111875, rs4402960, rs5215, rs1801282. For each of these SNPs, an odds ratio for three possible genotypes is reported in the literature. The CEU population consists of thirty mother-father-child trios. Sixty parents from this population are used in order to avoid dependencies. One of the individuals that had a no-call in one of the 10 SNPs is excluded, resulting in a set of 59 individuals. The GCI rank for each of the individuals is then calculated using several different models.

Different models produce highly correlated results for this dataset. The Spearman correlation is calculated between each pair of models (Table 2), which shows that the Multiplicative and Additive model has a correlation coefficient of 0.97, and thus the GCI score is robust using either the additive or multiplicative models. Similarly, the correlation between the Harvard modified scores and the multiplicative model is 0.83, and the correlation coefficient between the Harvard scores and the additive model is 0.7. However, using the maximum odds ratio as the genetic score yields a dichotomous score which is defined by one SNP. Overall these results indicate score ranking provides a robust framework that minimized model dependency.

TABLE 2
The Spearman correlations for the score distributions
on the CEU data between model pairs.

| | | | | | MAX |
|---|---|---|---|---|---|
| | Multiplicative | Additive | Harv-Het | Harv-Hom | OR |
| Mult | 1 | 0.97 | 0.83 | 0.83 | 0.42 |
| Additive | 0.97 | 1. | 0.7 | 0.7 | 0.6 |
| Harv-Het | 0.83 | 0.7 | 1 | 1 | 0 |
| Harv-Hom | 0.83 | 0.7 | 1 | 1 | 0 |
| MAX OR | 0.42 | 0.6 | 0 | 0 | 1 |

The effect of variation in the prevalence of T2D on the resulting distribution was measured. The prevalence values from 0.001 to 0.512 was varied. For the case of T2D, it was observed that different prevalence values result in the same order of individuals (Spearman correlation>0.99), therefore an artificially fixed value of prevalence 0.01 could be presumed.

### Example 2

### Evaluation of GCI

The WTCCC data (Wellcome Trust Case Control Consortium, Nature. 447:661-678 (2007)) is used to test the GCI framework. This dataset contains the genotypes of approximately 14,000 individuals divided into eight populations. The eight populations consist of seven populations of cases carrying seven different diseases, and one control population. All individuals are genotyped using the Affymetrix 500k GeneChip. For 3 of the 7 different diseases, Type 2 Diabetes, Crohn's Disease, and Rheumatoid Arthritis, SNPs that pass the curation standard set is searched on the Affymetrix 500k GeneChip for a SNP with r²=1 to the original published SNP. 8 SNPs for Type 2 Diabetes, 9 SNPs for Crohn's Disease, and 5 SNPs for Rheumatoid Arthritis are found.

The Receiver Operating Curves (ROC) (The Statistical Evaluation of Medical Tests for Classification and Prediction, M S Pepe. Oxford Statistical Science Series, Oxford University Press (2003)) is used to evaluate the ability of the GCI to serve as a classifier test for a condition. Preferably, a threshold t would exist, such that if an individual's GCI score exceeds t then the individual is necessarily a case, and if the individual's GCI score is lower than t, then the individual is necessarily a control. The GCI score for every individual is calculated in the three case-control sets as described above. The true positive rate as a function of the false positive rate based on a binary test defined by GCI score threshold is then plotted. Finally, the Area Under the Curve (AUC) of the resulting graph is calculated. For a random diagnostic test the AUC is 0.5, and for a perfect test the AUC is 1.

In order to have a baseline for comparison, the logistic regression to calculate the best model that leverages interactions between the SNPs to fit the data is used. If the SNPs are s₁,s₂, . . . ,sₙ, then the model assumes that the logit is X=a₁s₁+a₂s₂+ . . . +aₙsₙ+a₁₂s₁₂+ . . . +a_{n-1,n}s_{n-1,n}, where sᵢⱼ is the interaction between sᵢ and sⱼ. The fitted probability is used as an estimate for the risk, and generates a ROC curve for these risk estimates. Note that this model takes into account pairwise interactions between the SNPs, and it should therefore be at least as accurate as the GCI score.

The AUC for the GCI and for the logistic regression are quite similar for all three diseases (Table 2), leading to the conclusion that SNP-SNP interactions do not add substantial information for the risk assessment, at least not for these diseases and these SNPs. Therefore, it can be justified that the assumption that the SNP-SNP interactions can be ignored as long as there is no evidence for such an interaction from previous studies.

The GCI ROC curve is compared to a theoretical disease model. This disease model assumes that the disease is affected by both environmental and genetic factors, and that the two factors are independent. P=G+E, where G is the genetic risk and E is the environmental risk. The first model assumes that G˜N(0, σ_{G}), and E˜N(0, σ_{E}), and that an individual will develop the condition in his lifetime if P>α for a fixed α. The σ_{G}, σ_{E}, and α is fixed using the constraint that the heritability is σ_{G}/(σ_{G}+σ_{E}), and that the average lifetime risk is Pr(P>α). Since the heritability and average lifetime risks are known for each of the conditions tested, the parameters of the models according to the disease can be set. 100000 random samples were generated from the distribution P based on this model. It is then assumed that G is known for each individual (but E is not known, and therefore the disease status is unknown), and a ROC curve based on G is generated. This represents the optimal scenario where the genetic risk is entirely understood and can be measured for every individual.

A variant of this model in which G=λX+Y, where Y˜N(0, σ_{Y}), and X˜B(2,p) is also generated. In this case, X corresponds to one SNP with a large effect, and Y corresponds to many other small genetic effects. By setting the parameters λ, σ_{Y}, and p appropriately, the relative risks of the large effect SNP can be controlled. These relative risks are to be 4 for the risk-risk genotype, and 2 for the heterozygous were set.

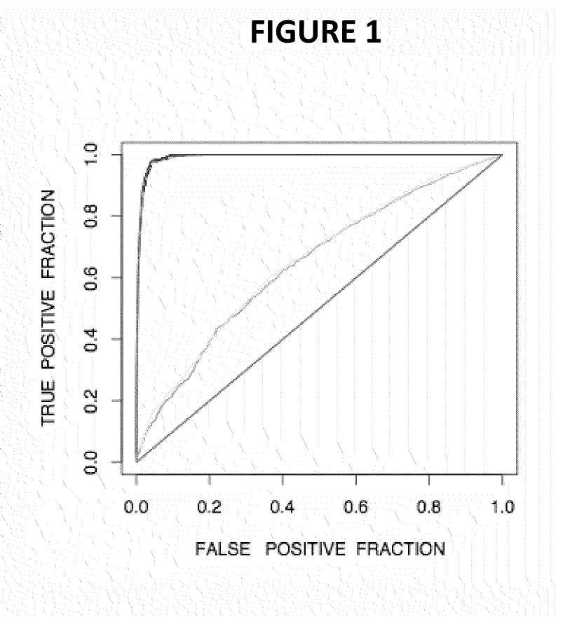

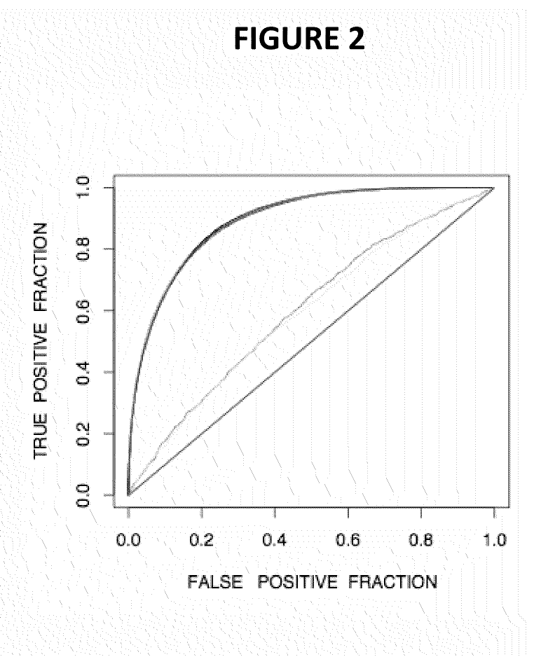

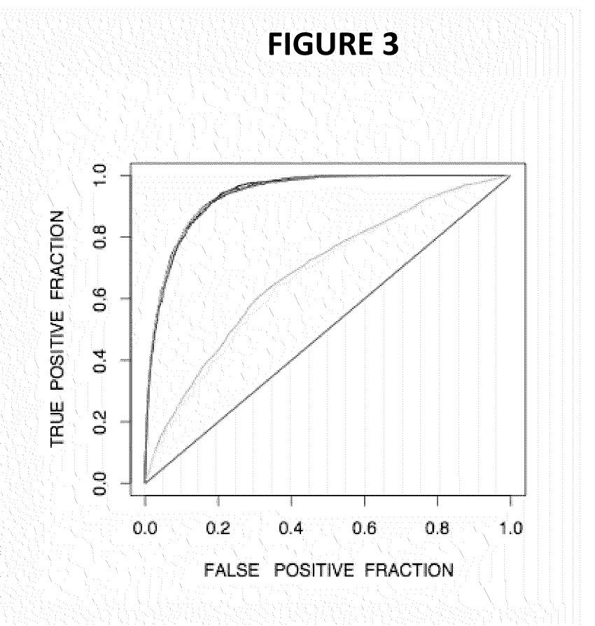

As can be seen by Table 3 and FIGS. 1-3, the AUC of the logistic regression and the GCI are quite close, and they are both bounded away from the random test. However, it is apparent that the theoretically optimal scenarios are more informative than our current estimates. Based on these graphs, current scientific knowledge enables the estimation of individual risk for disease in an informative way; as evidence, the AUC for the GCI is 20-40% higher than the random test that uses no information.

TABLE 3
The Area Under the Curve (AUC) for the different ROC curves.

| | | Average | Optimal | | Logistic |
|---|---|---|---|---|---|
| | Herit- | lifetime | Scenario | | Regression |
| Disease | ability | risk | AUC | GCI AUC | AUC |
| Type 2 | 64% | 25% | 0.902 | 0.596750 | 0.603873 |
| Diabetes | | | | | |
| Crohn's | 80% | 0.56% | 0.982 | 0.654024 | 0.646273 |
| Disease | | | | | |
| Rheumatoid | 53% | 1.54% | 0.944 | 0.674906 | 0.688608 |
| Arthritis | | | | | |

### Example 3

### Personalized Action Plan

A genomic profile is obtained from a saliva sample and a phenotype profile with GCI scores is generated. The report also includes a personalized action plan with recommendations as shown in Table 4.

TABLE 4
Personalized Action Plan.

| | Mod. | Wt. | | 8 Hr. | Consider | Increase | Avoid | Antiox. & |
|---|---|---|---|---|---|---|---|---|
| | exercise | Reduction | Aspirin | sleep | Statin | fiber | gluten | Omega3 |
| Myocardial | Y | Y | Y | Y | Y | Y | N | Y |
| infarction | | | | | | | | |
| Celiac disease | N | N | N | N | N | N | Y | N |
| Colon cancer | Y | Y | Y | N | N | Y | N | Y |
| Diabetes | Y | Y | N | N | Y | Y | N | N |
| Obesity | Y | Y | N | Y | N | Y | N | N |
| Alzheimer's | Y | N | Y | Y | Y | N | N | Y |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the disclosure described herein may be employed in practicing the embodiments. It is intended that the following claims define the scope of the disclosure and that methods and structures within the scope of these embodiments and their equivalents be covered thereby.

What is claimed is:

1. A rating system for a variety of recommendations in a personalized action plan,
wherein each of said recommendations is given a rating,
wherein each of said ratings corresponds to a rating given to an individual,
wherein said rating given to said individual is determined by a computer based on a genomic profile of said individual.

2. A rating system for a variety of recommendations in a personalized action plan,
wherein each of said recommendations is given a rating,
wherein each of said ratings corresponds to a rating given to an individual,
wherein said rating given to said individual is determined by a computer based on a Genetic Composite Index (GCI) or GCI Plus score of said individual.

3. The rating system of claim 1 or 2, wherein said rating is a number, color, letter, or combination thereof.

4. The rating system of claim 1 or 2, wherein recommendations includes a pharmaceutical recommendation.

5. The rating system of claim 1 or 2, wherein said non-pharmaceutical recommendation is an exercise regimen.

6. The rating system of claim 1 or 2, wherein said non-pharmaceutical recommendation is an exercise activity.

7. The rating system of claim 1 or 2, wherein said non-pharmaceutical recommendation is a dietary plan.

8. The rating system of claim 1 or 2, wherein said non-pharmaceutical recommendation is a nutrient.

9. The rating system of claim 1 or 2, wherein said rating system is represented by a binary system.

10. The rating system of claim 1 or 2, wherein said genomic profile is obtained using a high density DNA microarray or PCR-based method.

11. The rating system of claim 1 or 2, wherein said genomic profile is obtained by amplifying a genetic sample from said individual.

12. A method of providing a rating for recommendations in a personalized action plan to an individual comprising:
(a) obtaining a genomic profile of said individual;
(b) determining at least one rating for said individual, wherein said rating is determined by a computer based on said genomic profile; and,
(c) reporting said rating outputted from said computer to said individual or health care manager of said individual.

13. A method of providing a rating for recommendations in a personalized action plan to an individual comprising:
(a) generating a GCI or GCI Plus score for said individual using a computer;
(b) determining at least one rating for said individual, wherein said rating is determined by said computer based on said GCI or GCI Plus score; and,
(c) reporting said rating outputted from said computer to said individual or health care manager of said individual.

14. The method of claim 12 or 13, wherein said rating is represented by a color, letter, or number.

15. The method of claim 12 or 13, wherein said rating corresponds to a recommendation on a personalized action plan.

16. The method of claim 12 or 13, wherein recommendations includes a pharmaceutical recommendation.

17. The method of claim 12 or 13, wherein said non-pharmaceutical recommendation is an exercise activity.

18. The method of claim 12 or 13, wherein said non-pharmaceutical recommendation is a dietary plan.

19. The method of claim 12 or 13, wherein said non-pharmaceutical recommendation is a nutrient.

20. The method of claim 12 or 13, wherein said rating is based on a binary system.

21. The method of claim 12, wherein said genomic profile is obtained using a high density DNA microarray or PCR-based method.

22. The rating system of claim 12, wherein said genomic profile is obtained by amplifying a genetic sample from said individual.

23. A method for motivating an individual to improve their health comprising:
(a) obtaining a genomic profile for said individual;
(b) generating a personalized action plan for said individual using a computer;
(c) associating at least one incentive for said individual with an achievement of a recommendation on said personalized action plan generated from the computer in step (b); and,
(d) granting to said individual said incentive when said achievement is accomplished.

24. A method for motivating an individual to improve their health comprising:
(a) obtaining a genomic profile for said individual;
(b) generating at least one GCI or GCI Plus score for said individual using a computer;
(c) associating at least one incentive for said individual with an improvement of at least one GCI or GCI Plus score generated from said computer in step (b); and,
(d) granting to said individual said incentive when said improvement is achieved.

25. The method of claim 23 or 24, wherein said incentive is provided by an employer, friend, or family member.

26. The method of claim 23 or 24, wherein said individual is an employee.

27. The method of claim 26, wherein said incentive is a contribution by an employer of said individual to a health savings account, extra vacation days, or increased employer subsidy for said individual's medical plan.

28. The method of claim 23 or 24, wherein said incentive is cash.

29. The method of claim 23 or 24, wherein said incentive is a pharmaceutical product, health product, health club membership, medical follow-up, medical device, updated GCI or GCI Plus score, updated personalized action plan, or membership to an on-line community.

30. The method of claim 23 or 24, wherein said incentive is a discount, subsidy or reimbursement for a pharmaceutical product, health product, health club membership, medical follow-up, medical device, updated GCI or GCI Plus score, updated personalized action plan, or membership to an on-line community.

31. The method of claim 23 or 24, wherein said incentive is support through an on-line community.

32. The method of claim 23 or 24, wherein said genomic profile is obtained using a high density DNA microarray or PCR-based method.

33. The rating system of claim 23 or 24, wherein said genomic profile is obtained by amplifying a genetic sample from said individual.
